(19) United States
(12) Patent Application Publication
(10) Pub. No.: US 2025/0388564 A1
(43) Pub. Date: Dec. 25, 2025

(54) METHODS OF MANUFACTURING PYRIDAZINONE COMPOUNDS

(71) Applicant: Edgewise Therapeutics, Inc., Boulder, CO (US)

(72) Inventors: Kevin Koch, Niwot, CO (US); Kevin Hunt, Boulder, CO (US); Stephen Thomas Shclachter, Boulder, CO (US); Jonathan Lane, Longmont, CO (US); Todd Nelson, Cranbury, NJ (US); Christopher Kassl, Palatine, IL (US); Ana Cristina Parra Rivera, Cranbury, NJ (US); Chun-Min Zeng, South Plainfield, NJ (US); Anil Kumar, Cranbury, NJ (US); Thorsten Rosner, South Plainfield, NJ (US); Aurpon Mitra, Cranbury, NJ (US); Praveen Kilaru, South Plainfield, NJ (US)

(21) Appl. No.: 18/863,845

(22) PCT Filed: May 10, 2023
(86) PCT No.: PCT/US2023/021739

§ 371 Date: Nov. 7, 2024

Related U.S. Application Data
(60) Provisional application No. 63/340,871, filed on May 11, 2022.

(51) Int. Cl. C07D 401/14; A61K 31/501; A61K 31/69; C07D 213/26; C07D 237/14; C07D 401/06; C07F 5/02

(52) U.S. Cl. CPC C07D 401/14; A61K 31/501; A61K 31/69; C07D 213/26; C07D 237/14; C07D 401/06; C07F 5/025

(57) ABSTRACT

Disclosed herein are methods of manufacturing pyridazinone compounds useful in the treatment of Duchenne Muscular Dystrophy using Suzuki cross-coupling reactions.

### CROSS-REFERENCE

This application claims the benefit of U.S. Provisional Application Ser. No. 63/340,871 filed May 11, 2023, which is hereby incorporated by reference in its entirety.

### BACKGROUND

Duchenne Muscular Dystrophy (DMD) is a genetic disorder affecting skeletal muscle and is characterized by progressive muscle degeneration and weakness. There remains a need for treatments that reduce muscle breakdown in patients with neuromuscular conditions such as DMD.

### SUMMARY

Described herein are methods of making compounds for the treatment of DMD.

Disclosed herein is a process for the preparation of a compound of Formula 1:

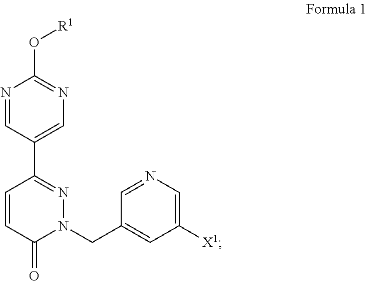

wherein X¹ is a halogen; and
R¹ is C₁-C₆ haloalkyl;
   comprising:
reacting a compound of Formula 2:

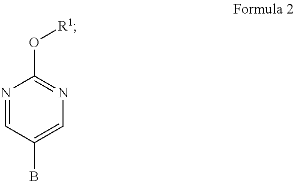

wherein R¹ is C₁-C₆ haloalkyl; and
B is selected from a boronic acid and a boronic ester;
with a compound of Formula 3 under coupling conditions:

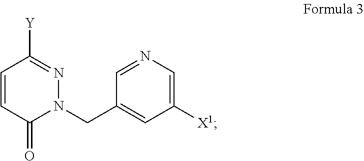

wherein X¹ is a halogen; and
Y is a leaving group;
in the presence of a metal catalyst, a suitable base, in a suitable solvent, to provide a compound of Formula 1.

Disclosed herein is a process for the preparation of a compound of Formula 2:

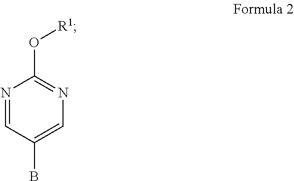

wherein R¹ is C₁-C₆ haloalkyl; and
B is selected from a boronic acid and boronic ester;
   comprising:
contacting a compound of Formula 6:

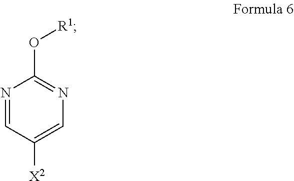

wherein R¹ is C₁-C₆ haloalkyl; and
X² is halogen;
with a boron compound, wherein the boron compound comprises a boron-boron bond or a boron-hydrogen bond;
in the presence of a metal catalyst, a suitable base, in a suitable solvent, to provide a compound of Formula 2.

### DETAILED DESCRIPTION

The present disclosure generally relates to methods of manufacturing a compound of Formula 1, such as 2-((5-fluoropyridin-3-yl)methyl)-6-(2-(2,2,2-trifluoroethoxy)pyrimidin-5-yl)pyridazin-3(2H)-one (Compound I). Compound I has been shown to treat neuromuscular conditions through selective inhibition of fast-fiber skeletal muscle myosin.

Skeletal muscle is mainly composed of two types of fibers, slow-twitch muscle fiber (i.e., type I) and fast-twitch muscle fiber (i.e., type II). In each muscle, the two types of fibers are configured in a mosaic-like arrangement, with differences in fiber type composition in different muscles and at different points in growth and development. Slow-twitch muscle fibers have excellent aerobic energy production ability. Contraction rate of the slow-twitch muscle fiber is low but tolerance to fatigue is high. Slow-twitch muscle fibers typically have a higher concentration of mitochondria and myoglobin than do fast-twitch fibers and are surrounded by more capillaries than are fast-twitch fibers. Slow-twitch fibers contract at a slower rate due to lower myosin ATPase activity and produce less power compared to fast-twitch fibers, but they are able to maintain contractile function over longer-terms, such as in stabilization, postural control, and endurance exercises.

Fast twitch muscle fibers in humans are further divided into two main fiber types depending on the specific fast skeletal myosin they express (Type IIa, IIx/d). A third type of fast fiber (Type IIb) exists in other mammals but is rarely identified in human muscle. Fast-twitch muscle fibers have excellent anaerobic energy production ability and are able to generate high amounts of tension over a short period of time. Typically, fast-twitch muscle fibers have lower concentrations of mitochondria, myoglobin, and capillaries compared to slow-twitch fibers, and thus can fatigue more quickly. Fast-twitch muscles produce quicker force required for power and resistance activities.

Inhibitors of skeletal muscle myosin that are not selective for the type II fibers may lead to excessive inhibition of skeletal muscle contraction including respiratory function and unwanted inhibition of cardiac activity as the heart shares several structural components (such as type I myosin) with type I skeletal muscle fibers. While not wishing to be bound by a particular mechanistic theory, this disclosure provides the manufacturing protocols and intermediates therein to generate selective inhibitors of fast-fiber skeletal muscle myosin as a treatment option for DMD and other neuromuscular conditions. The targeted inhibition of type II skeletal muscle myosin may reduce skeletal muscle contractions while minimizing the impact on a subject's daily activities.

### Preparation of Compound I

Disclosed herein are novel methods for the synthesis of a compound of Formula 1 such as Compound I. In some embodiments, a compound of Formula 1 is synthesized in Scheme 1.

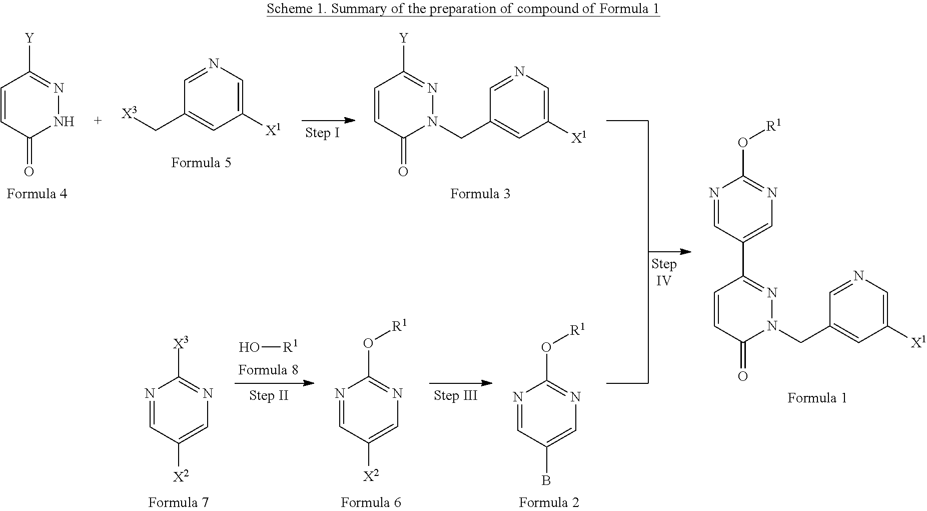

Briefly, in some embodiments, a compound of Formula 4 and Formula 5 is coupled to form Formula 3. In some embodiments, a compound of Formula 7 is treated with an alcohol of Formula 8 yield a compound of Formula 6. In some embodiments, a compound of Formula 6 is borylated with a suitable borylation agent under coupling conditions to form a compound of Formula 2. In some embodiments, a compound of Formula 3 is cross coupled with a compound of Formula 2 under coupling conditions to yield a compound of Formula 1. In some embodiments, a compound of Formula 2 is generated in situ and further reacted with a compound of Formula 3 under coupling conditions to form a compound of Formula 1. In some embodiments, a compound of Formula 2 is generated and isolated before further reaction with a compound of Formula 3 under coupling conditions to form a compound of Formula 1.

As disclosed herein, variables in Scheme 1 are defined as follows: R¹ is C₁-C₆ haloalkyl; X¹ is a halogen; X² is a halogen; X³ is a halogen; Y is a leaving group; and B is selected from a boronic acid and a boronic ester.

In some embodiments, R¹ is C₁-C₃ haloalkyl. In some embodiments, R¹ is selected from —CF₃, —CHF₂, —CH₂F, —CH₂CF₃, —CH₂CHF₂, and —CH₂CHF₂. In some embodiments, R¹ is selected from —CF₃, —CHF₂, —CH₂CF₃, and —CH₂CHF₂. In some embodiments, R¹ is selected from CHF₂ and —CH₂CF₃. In some embodiments, R¹ is —CH₂CF₃.

In some embodiments, X¹ is selected from —F, —Cl, and —Br. In some embodiments, X¹ is selected from —Cl, and —Br. In some embodiments, X¹ is selected from —F, and —Cl. In some embodiments, X¹ is —F. In some embodiments, X¹ is —Cl. In some embodiments, X¹ is —Br.

In some embodiments, X² is selected from —Cl, and —Br. In some embodiments, X² is —Cl. In some embodiments, X² is —Br.

In some embodiments, X³ is selected from —Cl and —Br. In some embodiments, X³ is —Cl. In some embodiments, X³ is —Br.

In some embodiments, Y is selected from halogen, and pseudo halide. In some embodiments, Y is selected from halogen, -OTf, -OTs, and -OMs. In some embodiments, Y is selected from halogen and -OTf. In some embodiments, Y is selected from —Cl, —Br, and —I. In some embodiments, Y is selected from —Cl, and —Br. In some embodiments, Y is —Cl. In some embodiments, Y is —Br. In some embodiments, Y is —I.

In some embodiments, B is selected from

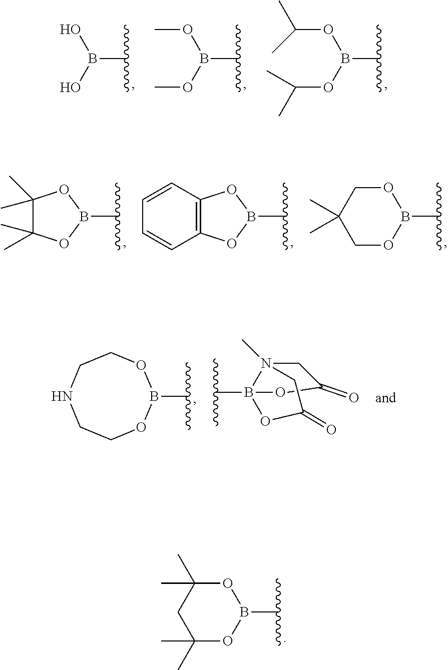

In some embodiments, B is selected from

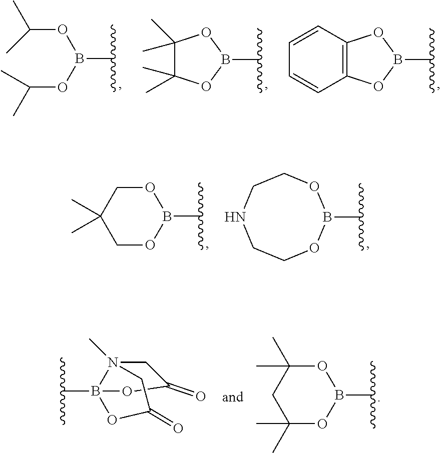

In some embodiments, B is selected from

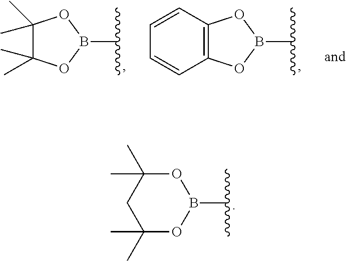

In some embodiments, B is

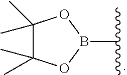 [SMILES: *B1OC(C)(C)C(C)(C)O1]

In some embodiments, B is

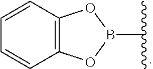 [SMILES: *B1Oc2ccccc2O1]

In some embodiments, the compound of Formula 1 is Compound I. In some embodiments, the compound of Formula 2 is Compound II. In some embodiments, the compound of Formula 3 is Compound III. In some embodiments, the compound of Formula 4 is Compound IV. In some embodiments, the compound of Formula 5 is Compound V. In some embodiments, the compound of Formula 6 is Compound VI. In some embodiments, the compound of Formula 7 is Compound VII. In some embodiments, the compound of Formula 8 is CF₃CH₂OH. Compounds I-VII are depicted below.

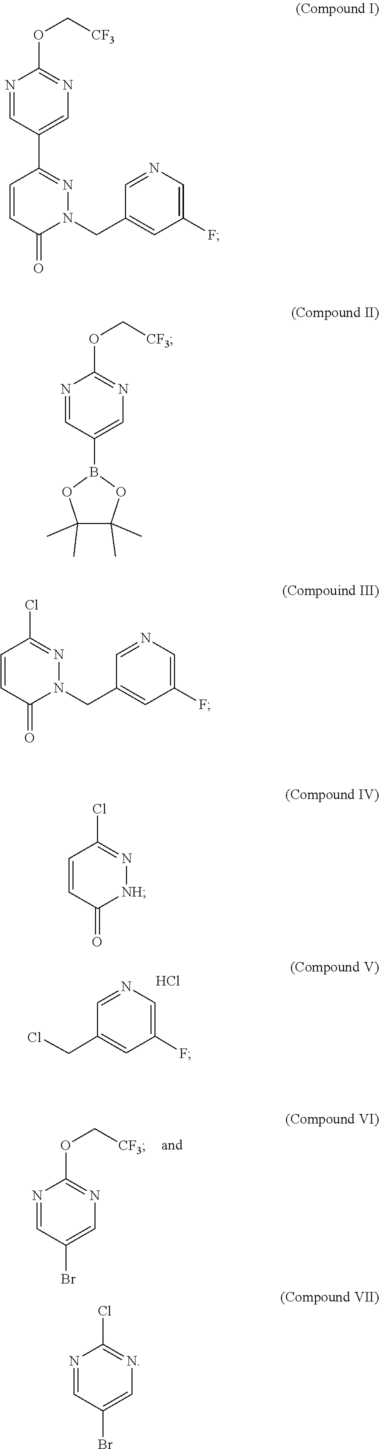

In some embodiments, the isolated yield of a compound of Formula 3 in Step I is about 50% to about 95%. In some embodiments, the isolated yield of a compound of Formula 3 in Step I is about 50% to about 55%, about 50% to about 65%, about 50% to about 70%, about 50% to about 72%, about 50% to about 74%, about 50% to about 76%, about 50% to about 78%, about 50% to about 80%, about 50% to about 85%, about 50% to about 90%, about 50% to about 95%, about 55% to about 65%, about 55% to about 70%, about 55% to about 72%, about 55% to about 74%, about 55% to about 76%, about 55% to about 78%, about 55% to about 80%, about 55% to about 85%, about 55% to about 90%, about 55% to about 95%, about 65% to about 70%, about 65% to about 72%, about 65% to about 74%, about 65% to about 76%, about 65% to about 78%, about 65% to about 80%, about 65% to about 85%, about 65% to about 90%, about 65% to about 95%, about 70% to about 72%, about 70% to about 74%, about 70% to about 76%, about 70% to about 78%, about 70% to about 80%, about 70% to about 85%, about 70% to about 90%, about 70% to about 95%, about 72% to about 74%, about 72% to about 76%, about 72% to about 78%, about 72% to about 80%, about 72% to about 85%, about 72% to about 90%, about 72% to about 95%, about 74% to about 76%, about 74% to about 78%, about 74% to about 80%, about 74% to about 85%, about 74% to about 90%, about 74% to about 95%, about 76% to about 78%, about 76% to about 80%, about 76% to about 85%, about 76% to about 90%, about 76% to about 95%, about 78% to about 80%, about 78% to about 85%, about 78% to about 90%, about 78% to about 95%, about 80% to about 85%, about 80% to about 90%, about 80% to about 95%, about 85% to about 90%, about 85% to about 95%, or about 90% to about 95%. In some embodiments, the isolated yield of a compound of Formula 3 in Step I is about 50%, about 55%, about 65%, about 70%, about 72%, about 74%, about 76%, about 78%, about 80%, about 85%, about 90%, or about 95%. In some embodiments, the isolated yield of a compound of Formula 3 in Step I is at least about 50%, about 55%, about 65%, about 70%, about 72%, about 74%, about 76%, about 78%, about 80%, about 85%, or about 90%. In some embodiments, the isolated yield of a compound of Formula 3 in Step I is at most about 55%, about 65%, about 70%, about 72%, about 74%, about 76%, about 78%, about 80%, about 85%, about 90%, or about 95%.

In some embodiments, the isolated yield of a compound of Formula 6 in Step II is about 70% to about 99%. In some embodiments, the isolated yield of a compound of Formula 6 in Step II is about 70% to about 80%, about 70% to about 90%, about 70% to about 91%, about 70% to about 92%, about 70% to about 93%, about 70% to about 94%, about 70% to about 95%, about 70% to about 96%, about 70% to about 97%, about 70% to about 98%, about 70% to about 99%, about 80% to about 90%, about 80% to about 91%, about 80% to about 92%, about 80% to about 93%, about 80% to about 94%, about 80% to about 95%, about 80% to about 96%, about 80% to about 97%, about 80% to about 98%, about 80% to about 99%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 91% to about 92%, about 91% to about 93%, about 91% to about 94%, about 91% to about 95%, about 91% to about 96%, about 91% to about 97%, about 91% to about 98%, about 91% to about 99%, about 92% to about 93%, about 92% to about 94%, about 92% to about 95%, about 92% to about 96%, about 92% to about 97%, about 92% to about 98%, about 92% to about 99%, about 93% to about 94%, about 93% to about 95%, about 93% to about 96%, about 93% to about 97%, about 93% to about 98%, about 93% to about 99%, about 94% to about 95%, about 94% to about 96%, about 94% to about 97%, about 94% to about 98%, about 94% to about 99%, about 95% to about 96%, about 95% to about 97%, about 95% to about 98%, about 95% to about 99%, about 96% to about 97%, about 96% to about 98%, about 96% to about 99%, about 97% to about 98%, about 97% to about 99%, or about 98% to about 99%. In some embodiments, the isolated yield of a compound of Formula 6 in Step II is about 70%, about 80%, about 90%, about 91%, about 92%, about 93%, about 94%, about 95%, about 96%, about 97%, about 98%, or about 99%. In some embodiments, the isolated yield of a compound of Formula 6 in Step II is at least about 70%, about 80%, about 90%, about 91%, about 92%, about 93%, about 94%, about 95%, about 96%, about 97%, or about 98%. In some embodiments, the isolated yield of a compound of Formula 6 in Step II is at most about 80%, about 90%, about 91%, about 92%, about 93%, about 94%, about 95%, about 96%, about 97%, about 98%, or about 99%.

In some embodiments, the unisolated yield of a compound of Formula 6 in Step II is about 70% to about 99%. In some embodiments, the unisolated yield of a compound of Formula 6 in Step II is about 70% to about 80%, about 70% to about 90%, about 70% to about 91%, about 70% to about 92%, about 70% to about 93%, about 70% to about 94%, about 70% to about 95%, about 70% to about 96%, about 70% to about 97%, about 70% to about 98%, about 70% to about 99%, about 80% to about 90%, about 80% to about 91%, about 80% to about 92%, about 80% to about 93%, about 80% to about 94%, about 80% to about 95%, about 80% to about 96%, about 80% to about 97%, about 80% to about 98%, about 80% to about 99%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 91% to about 92%, about 91% to about 93%, about 91% to about 94%, about 91% to about 95%, about 91% to about 96%, about 91% to about 97%, about 91% to about 98%, about 91% to about 99%, about 92% to about 93%, about 92% to about 94%, about 92% to about 95%, about 92% to about 96%, about 92% to about 97%, about 92% to about 98%, about 92% to about 99%, about 93% to about 94%, about 93% to about 95%, about 93% to about 96%, about 93% to about 97%, about 93% to about 98%, about 93% to about 99%, about 94% to about 95%, about 94% to about 96%, about 94% to about 97%, about 94% to about 98%, about 94% to about 99%, about 95% to about 96%, about 95% to about 97%, about 95% to about 98%, about 95% to about 99%, about 96% to about 97%, about 96% to about 98%, about 96% to about 99%, about 97% to about 98%, about 97% to about 99%, or about 98% to about 99%. In some embodiments, the unisolated yield of a compound of Formula 6 in Step II is about 70%, about 80%, about 90%, about 91%, about 92%, about 93%, about 94%, about 95%, about 96%, about 97%, about 98%, or about 99%. In some embodiments, the unisolated yield of a compound of Formula 6 in Step II is at least about 70%, about 80%, about 90%, about 91%, about 92%, about 93%, about 94%, about 95%, about 96%, about 97%, or about 98%. In some embodiments, the unisolated yield of a compound of Formula 6 in Step II is at most about 80%, about 90%, about 91%, about 92%, about 93%, about 94%, about 95%, about 96%, about 97%, about 98%, or about 99%.

In some embodiments, the isolated yield of a compound of Formula 2 in Step III is about 50% to about 85%. In some embodiments, the isolated yield of a compound of Formula 2 in Step III is about 50% to about 55%, about 50% to about 60%, about 50% to about 62%, about 50% to about 64%, about 50% to about 66%, about 50% to about 68%, about 50% to about 70%, about 50% to about 73%, about 50% to about 75%, about 50% to about 80%, about 50% to about 85%, about 55% to about 60%, about 55% to about 62%, about 55% to about 64%, about 55% to about 66%, about 55% to about 68%, about 55% to about 70%, about 55% to about 73%, about 55% to about 75%, about 55% to about 80%, about 55% to about 85%, about 60% to about 62%, about 60% to about 64%, about 60% to about 66%, about 60% to about 68%, about 60% to about 70%, about 60% to about 73%, about 60% to about 75%, about 60% to about 80%, about 60% to about 85%, about 62% to about 64%, about 62% to about 66%, about 62% to about 68%, about 62% to about 70%, about 62% to about 73%, about 62% to about 75%, about 62% to about 80%, about 62% to about 85%, about 64% to about 66%, about 64% to about 68%, about 64% to about 70%, about 64% to about 73%, about 64% to about 75%, about 64% to about 80%, about 64% to about 85%, about 66% to about 68%, about 66% to about 70%, about 66% to about 73%, about 66% to about 75%, about 66% to about 80%, about 66% to about 85%, about 68% to about 70%, about 68% to about 73%, about 68% to about 75%, about 68% to about 80%, about 68% to about 85%, about 70% to about 73%, about 70% to about 75%, about 70% to about 80%, about 70% to about 85%, about 73% to about 75%, about 73% to about 80%, about 73% to about 85%, about 75% to about 80%, about 75% to about 85%, or about 80% to about 85%. In some embodiments, the isolated yield of a compound of Formula 2 in Step III is about 50%, about 55%, about 60%, about 62%, about 64%, about 66%, about 68%, about 70%, about 73%, about 75%, about 80%, or about 85%. In some embodiments, the isolated yield of a compound of Formula 2 in Step III is at least about 50%, about 55%, about 60%, about 62%, about 64%, about 66%, about 68%, about 70%, about 73%, about 75%, or about 80%. In some embodiments, the isolated yield of a compound of Formula 2 in Step III is at most about 55%, about 60%, about 62%, about 64%, about 66%, about 68%, about 70%, about 73%, about 75%, about 80%, or about 85%.

In some embodiments, the isolated yield of a compound of Formula I in Step IV is about 60% to about 95%. In some embodiments, the isolated yield of a compound of Formula I in Step IV is about 60% to about 65%, about 60% to about 70%, about 60% to about 73%, about 60% to about 76%, about 60% to about 78%, about 60% to about 80%, about 60% to about 82%, about 60% to about 85%, about 60% to about 88%, about 60% to about 90%, about 60% to about 95%, about 65% to about 70%, about 65% to about 73%, about 65% to about 76%, about 65% to about 78%, about 65% to about 80%, about 65% to about 82%, about 65% to about 85%, about 65% to about 88%, about 65% to about 90%, about 65% to about 95%, about 70% to about 73%, about 70% to about 76%, about 70% to about 78%, about 70% to about 80%, about 70% to about 82%, about 70% to about 85%, about 70% to about 88%, about 70% to about 90%, about 70% to about 95%, about 73% to about 76%, about 73% to about 78%, about 73% to about 80%, about 73% to about 82%, about 73% to about 85%, about 73% to about 88%, about 73% to about 90%, about 73% to about 95%, about 76% to about 78%, about 76% to about 80%, about 76% to about 82%, about 76% to about 85%, about 76% to about 88%, about 76% to about 90%, about 76% to about 95%, about 78% to about 80%, about 78% to about 82%, about 78% to about 85%, about 78% to about 88%, about 78% to about 90%, about 78% to about 95%, about 80% to about 82%, about 80% to about 85%, about 80% to about 88%, about 80% to about 90%, about 80% to about 95%, about 82% to about 85%, about 82% to about 88%, about 82% to about 90%, about 82% to about 95%, about 85% to about 88%, about 85% to about 90%, about 85% to about 95%, about 88% to about 90%, about 88% to about 95%, or about 90% to about 95%. In some embodiments, the isolated yield of a compound of Formula I in Step IV is about 60%, about 65%, about 70%, about 73%, about 76%, about 78%, about 80%, about 82%, about 85%, about 88%, about 90%, or about 95%. In some embodiments, the isolated yield of a compound of Formula I in Step IV is at least about 60%, about 65%, about 70%, about 73%, about 76%, about 78%, about 80%, about 82%, about 85%, about 88%, or about 90%. In some embodiments, the isolated yield of a compound of Formula I in Step IV is at most about 65%, about 70%, about 73%, about 76%, about 78%, about 80%, about 82%, about 85%, about 88%, about 90%, or about 95%.

In some embodiments, the overall isolated yield of a compound of Formula 1 starting from a compound of Formula 7 is about 35% to about 65%. In some embodiments, the overall isolated yield of a compound of Formula 1 starting from a compound of Formula 7 is about 35% to about 40%, about 35% to about 45%, about 35% to about 50%, about 35% to about 55%, about 35% to about 60%, about 35% to about 65%, about 40% to about 45%, about 40% to about 50%, about 40% to about 55%, about 40% to about 60%, about 40% to about 65%, about 45% to about 50%, about 45% to about 55%, about 45% to about 60%, about 45% to about 65%, about 50% to about 55%, about 50% to about 60%, about 50% to about 65%, about 55% to about 60%, about 55% to about 65%, or about 60% to about 65%. In some embodiments, the overall isolated yield of a compound of Formula 1 starting from a compound of Formula 7 is about 35%, about 40%, about 45%, about 50%, about 55%, about 60%, or about 65%. In some embodiments, the overall isolated yield of a compound of Formula 1 starting from a compound of Formula 7 is at least about 35%, about 40%, about 45%, about 50%, about 55%, or about 60%. In some embodiments, the overall isolated yield of a compound of Formula 1 starting from a compound of Formula 7 is at most about 40%, about 45%, about 50%, about 55%, about 60%, or about 65%.

### Step I: Preparation of a Compound of Formula 3

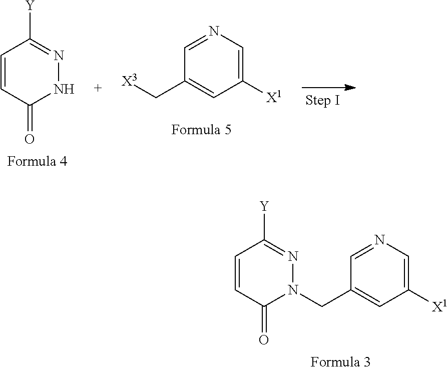

In some embodiments, a compound of Formula 4 and Formula 5 react in the presence of a suitable base, in a suitable solvent, to provide a compound of Formula 3.

In some embodiments, the compound of Formula 5 is an acidic salt. In some embodiments, the compound of Formula 5 is an HCl, HBr, HNO₃, or H₂SO₄ salt. In some embodiments, the compound of Formula 5 is an HCl salt.

In some embodiments, the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF. In some embodiments, the suitable base is selected from KOAc, NaHCO₃, and K₂CO₃. In some embodiments, the suitable base is selected from NaHCO₃, K₂CO₃, and Cs₂CO₃. In some embodiments, the suitable base is K₂CO₃. In some embodiments, the suitable base is K₂CO₃. In some embodiments, the suitable base is Cs₂CO₃.

In some embodiments, the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, methyl-t-butyl ether, water, and any combination thereof. In some embodiments, the suitable solvent is selected from N-methyl-2-pyrrolidone, tetrahydrofuran, methyl-t-butyl ether, tetrahydropyran, 1,4-dioxane, 2-methyltetrahydrofuran, water, and any combination thereof. In some embodiments, the suitable solvent is a combination of N-methyl-2-pyrrolidone and water.

In some embodiments, the reaction conditions comprise a stir time of about 0.1 h to about 24 h. In some embodiments, the reaction conditions comprise a stir time of about 0.1 h to about 12 h. In some embodiments, the reaction conditions comprise a stir time of about 0.5 h to about 5 h. In some embodiments, the reaction conditions comprise a stir time of about 3 h to about 5 h.

In some embodiments, the reaction conditions comprise a reaction temperature of about 10° C. to about 50° C. In some embodiments, the reaction conditions comprise a reaction temperature of about 10° C. to about 40° C. In some embodiments, the reaction conditions comprise a reaction temperature of about 15° C. to about 30° C. In some embodiments, the reaction conditions comprise a reaction temperature of about 20° C. to about 30° C. In some embodiments, the reaction conditions comprise a reaction temperature of about 20° C. to about 25° C.

In some embodiments, the compound of Formula 3 is Compound III, the compound of Formula 4 is Compound IV, and the compound of Formula 5 is Compound V. In some embodiments, the molar ratio of Compound IV to Compound V is from about 1.0:1.0 to about 1.0:1.5. In some embodiments, the molar ratio of Compound IV to Compound V is from about 1.0:1.0 to about 1:1.2. In some embodiments, the molar ratio of Compound IV to Compound V is about 1.0:1.0. In some embodiments, the suitable base is K₂CO₃. In some embodiments, the molar ratio of Compound IV to the suitable base is from about 1.0:5.0 to about 1.0:1.0. In some embodiments, the molar ratio of Compound IV to the suitable base is from about 1.0:4.0 to about 1.0:2.0. In some embodiments, the molar ratio of Compound IV to the suitable base is about 1.0:3.0.

### Step II: Preparation of a Compound of Formula 6

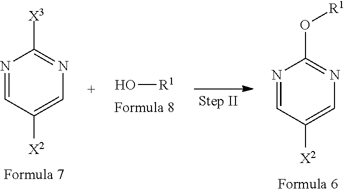

In some embodiments, a compound of Formula 7 reacts with alcohol of Formula 8 in the presence of a suitable base, in a suitable solvent, to provide a compound of Formula 6.

In some embodiments, the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF. In some embodiments, the suitable base is selected from KOAc, NaHCO₃, and K₂CO₃. In some embodiments, the suitable base is K₂CO₃.

In some embodiments, the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, methyl-t-butyl ether, water, and any combination thereof. In some embodiments, the suitable solvent is selected from dimethylformamide, N-methyl-2-pyrrolidone, tetrahydrofuran, methyl-t-butyl ether, tetrahydropyran, 1,4-dioxane, 2-methyltetrahydrofuran, water, and any combination thereof. In some embodiments, the suitable solvent is dimethylformamide.

In some embodiments, the reaction conditions comprise a stir time of about 0.1 h to about 24 h. In some embodiments, the reaction conditions comprise a stir time of about 0.1 h to about 12 h. In some embodiments, the reaction conditions comprise a stir time of about 0.5 h to about 5 h. In some embodiments, the reaction conditions comprise a stir time of about 3 h to about 5 h.

In some embodiments, the reaction conditions comprise a reaction temperature of about 10° C. to about 50° C. In some embodiments, the reaction conditions comprise a reaction temperature of about 10° C. to about 40° C. In some embodiments, the reaction conditions comprise a reaction temperature of about 15° C. to about 30° C. In some embodiments, the reaction conditions comprise a reaction temperature of about 20° C. to about 30° C. In some embodiments, the reaction conditions comprise a reaction temperature of about 20° C. to about 25° C.

In some embodiments, the compound of Formula 6 is Compound VI, the compound of Formula 7 is Compound VII and the compound of Formula 8 is CF 3CH₂OH. In some embodiments, the molar ratio of Compound VII to CF₃CH₂OH is from about 1.0:1.0 to about 1.0:1.5. In some embodiments, the molar ratio of Compound VII to CF₃CH₂OH is from about 1.0:1.0 to about 1:1.2. In some embodiments, the molar ratio of Compound VII to CF₃CH₂OH is about 1.0:1.1. In some embodiments, the suitable base is K₂CO₃. In some embodiments, the molar ratio of Compound VII to the suitable base is from about 1.0:5.0 to about 1.0:1.0. In some embodiments, the molar ratio of Compound VII to the suitable base is from about 1.0:4.0 to about 1.0:1.0. In some embodiments, the molar ratio of Compound VII to the suitable base is about 1.0:1.6.

In some embodiments, a compound of Formula 6 is not isolated prior to use in subsequent steps. In some embodiments, the compound of Formula 6 is carried forward in a solution used in step III. In some embodiments, water is removed from the solution of Formula 6 prior to use in Step III.

### Step III: Preparation of a Compound of Formula 2

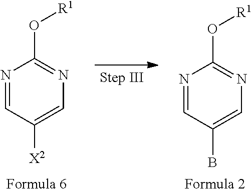

In some embodiments, a compound of Formula 6 is borylated with a boron compound in the presence of a metal catalyst, a suitable base, in a suitable solvent, to provide a compound of Formula 2.

In some embodiments, the boron compound comprises a boron-boron bond or a boron-hydrogen bond. In some embodiments, the boron compound is selected from

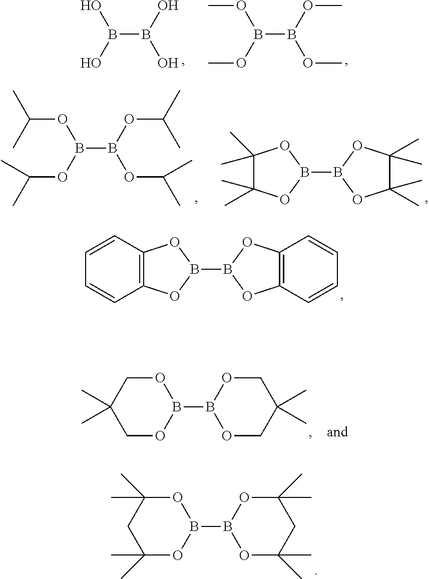

In some embodiments, the boron compound is selected from

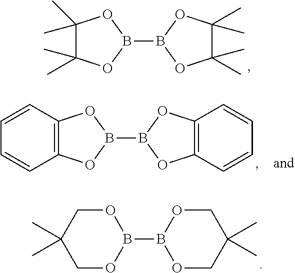

In some embodiments, the boron compound is

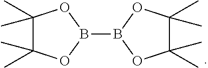

In some embodiments, the boron compound is

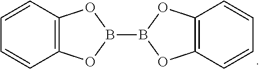 [SMILES: c1ccc2c(c1)OB(B1Oc3ccccc3O1)O2]

In some embodiments, the boron compound is

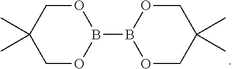

In some embodiments, the boron compound is

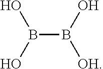

In some embodiments, B is selected from

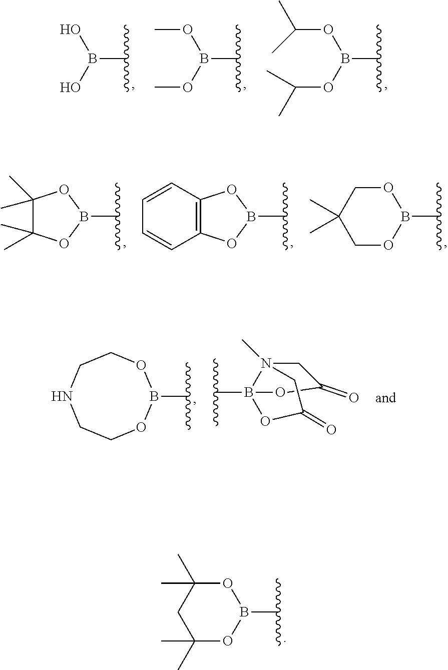

In some embodiments, B is selected from

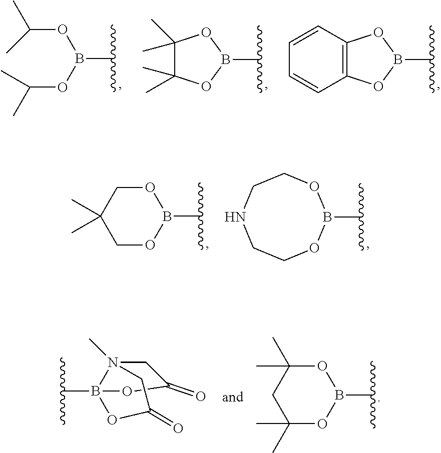

In some embodiments, B is selected from

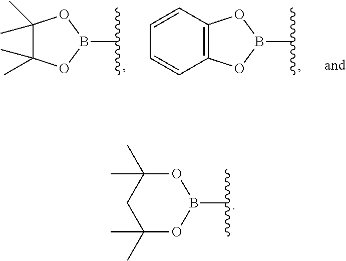

In some embodiments, B is

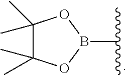 [SMILES: *B1OC(C)(C)C(C)(C)O1]

In some embodiments, B is

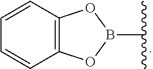 [SMILES: *B1Oc2ccccc2O1]

In some embodiments, the metal catalyst is suitable for Suzuki cross couplings. In some embodiments, the metal catalyst is a palladium catalyst. In some embodiments, the metal catalyst is selected from a palladium (0) or palladium (II) catalyst. In some embodiments, the metal catalyst comprises palladium and one or more ligand. In some embodiments, the ligand is selected from an N-heterocyclic carbene, a phosphine, a phosphite, and a bis-phosphine. In some embodiments, the ligand is selected from a phosphine, a phosphite, and a bis-phosphine. In some embodiments, the ligand is selected from a phosphine and a bis-phosphine.

In some embodiments, the phosphine is selected from trimethyl phosphine, tricyclohexylphosphine, tri-(tert-butyl)-phosphine, XantPhos, DPEPhos, XPhos, SPhos, JohnPhos, Cy-JohnPhos, Amphos, triphenylphosphine, methyldiphenylphosphine, Me4 t-BuXphos, t-BuXPhos, t-BuXantPhos, RuPhos, DavePhos, sSPhos, AdBrettPhos, BrettPhos, JackiePhos, t-BuBrettPhos, TrixiePos, t-BuDavePhos, t-BuMePhos, MePhos, PhDavePhos, VPhos, PhCPhos, XPhos-SO₃Na, water soluble SPhos, CPhos, EtCPhos, RockPhos, AlPhos, t-Bu PhCPhos, AlPhos. In some embodiments, the phosphine is selected from tricyclohexylphosphine, XantPhos, DPEPhos, XPhos, SPhos, Cy-JohnPhos, Amphos, and PhDavePhos.

In some embodiments, the phosphite is selected from trimethylphosphite and triphenylphosphite.

In some embodiments, the bis-phosphine is selected from bis(diphenylphosphino)methane (dppm), 1,2′-bis(diphenyl phosphino)ethane (dppe), 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf). In some embodiments, the bis-phosphine is selected from 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf).

In some embodiments, the metal catalyst is a selected from Pd(dppf)Cl₂, Pd(Amphos)₂Cl₂, Pd(dcypf)Cl₂, Pd(dtbpf)Cl₂, Pd(XantPhos)Cl₂, PdCl₂(DPEPhos), Pd(PCy₃)Cl₂, XPhosPd G2, and RuPhos-Pd-G2. In some embodiments, the metal catalyst is Pd(dppf)Cl₂. In some embodiments, the metal catalyst is Pd(Amphos)₂Cl₂.

In some embodiments, the metal catalyst is a palladacycle. In some embodiments, the metal catalyst is formed in solution.

In some embodiments, the metal catalysts described herein have two monodentate phosphine ligands. In some embodiments, the active metal catalyst may de-coordinate one of the monodentate phosphine ligands and/or form a palladacycle. For example, the compound Pd(Amphos)₂Cl₂ is envisioned to encompass Pd(Amphos)Cl₂ and/or the equivalent palladacycle.

In some embodiments, the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF. In some embodiments, the suitable base is selected from KOAc, NaHCO₃, and K₂CO₃. In some embodiments, the suitable base is K₂CO₃. In some embodiments, the suitable base is KOAc.

In some embodiments, the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, methyl-t-butyl ether, water, and any combination thereof. In some embodiments, the suitable solvent is selected from dimethylformamide, N-methyl-2-pyrrolidone, tetrahydrofuran, methyl-t-butyl ether, tetrahydropyran, 1,4-dioxane, 2-methyltetrahydrofuran, water, and any combination thereof. In some embodiments, the suitable solvent is 2-methyltetrahydrofuran.

In some embodiments, the reaction conditions comprise a stir time of about 0.5 h to about 48 h. In some embodiments, the reaction conditions comprise a stir time of about 1 h to about 36 h. In some embodiments, the reaction conditions comprise a stir time of about 5 h to about 30 h. In some embodiments, the reaction conditions comprise a stir time of about 10 h to about 20 h.

In some embodiments, the reaction conditions comprise a reaction temperature of about 50° C. to about 120° C. In some embodiments, the reaction conditions comprise a reaction temperature of about 60° C. to about 110° C. In some embodiments, the reaction conditions comprise a reaction temperature of about 60° C. to about 100° C. In some embodiments, the reaction conditions comprise a reaction temperature of about 70° C. to about 90° C. In some embodiments, the reaction conditions comprise a reaction temperature of about 80° C. to about 85° C.

In some embodiments, the compound of Formula 2 is Compound II, the compound of Formula 6 is Compound VI, and the boron compound is

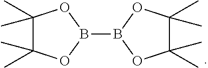

In some embodiments, the molar ratio of Compound VI to the boron compound is from about 1.0:1.0 to about 1.0:1.5. In some embodiments, the molar ratio of Compound VI to the boron compound is from about 1.0:1.0 to about 1:1.2. In some embodiments, the molar ratio of Compound VI to the boron compound is about 1.0:1.0. In some embodiments, the suitable base is KOAc. In some embodiments, the molar ratio of Compound VI to the suitable base is from about 1.0:5.0 to about 1.0:1.0. In some embodiments, the molar ratio of Compound VI to the suitable base is from about 1.0:4.0 to about 1.0:2.0. In some embodiments, the molar ratio of Compound VI to the suitable base is about 1.0:3.0. In some embodiments, the metal catalyst is Pd(dppf)₂Cl₂. In some embodiments, the metal catalyst is Pd(Amphos)₂Cl. In some embodiments, the molar ratio of Compound VI to the metal catalyst is between about 1.0:0.0001 to about 1.0:0.1. In some embodiments, the molar ratio of the Compound VI to the metal catalyst is between about 1.0:0.001 to about 1.0:0.05. In some embodiments, the molar ratio of the Compound VI to the metal catalyst is between about 1.0:0.05 to about 1.0:0.04. In some embodiments, the molar ratio of the Compound VI to the metal catalyst is between about 1.0:0.01 to about 1.0:0.02. In some embodiments, the molar ratio of the Compound VI to the metal catalyst is about 1.0:0.015.

In some embodiments, water is removed from the solution of Formula 6 prior to the addition of the catalyst and boron compound. In some embodiments, following water removal, the homocoupling impurity of Formula 6 is decreased. In some embodiments, the homocoupled impurity is decreased by about 1% to about 15%. In some embodiments, the homocoupled impurity is decreased by about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 3% to about 4%, about 3% to about 5%, about 3% to about 6%, about 3% to about 7%, about 3% to about 8%, about 3% to about 9%, about 3% to about 10%, about 3% to about 15%, about 4% to about 5%, about 4% to about 6%, about 4% to about 7%, about 4% to about 8%, about 4% to about 9%, about 4% to about 10%, about 4% to about 15%, about 5% to about 6%, about 5% to about 7%, about 5% to about 8%, about 5% to about 9%, about 5% to about 10%, about 5% to about 15%, about 6% to about 7%, about 6% to about 8%, about 6% to about 9%, about 6% to about 10%, about 6% to about 15%, about 7% to about 8%, about 7% to about 9%, about 7% to about 10%, about 7% to about 15%, about 8% to about 9%, about 8% to about 10%, about 8% to about 15%, about 9% to about 10%, about 9% to about 15%, or about 10% to about 15%. In some embodiments, the homocoupled impurity is decreased by about 1%, about 2%, about 3%, about 4%, about 5%, about 6%, about 7%, about 8%, about 9%, about 10%, or about 15%. In some embodiments, the homocoupled impurity is decreased by at least about 1%, about 2%, about 3%, about 4%, about 5%, about 6%, about 7%, about 8%, about 9%, or about 10%. In some embodiments, the homocoupled impurity is decreased by at most about 2%, about 3%, about 4%, about 5%, about 6%, about 7%, about 8%, about 9%, about 10%, or about 15%.

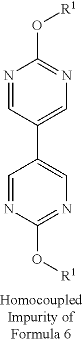

In some embodiments, water is removed from the solution of Formula 6 prior to the addition of the catalyst and boron compound. In some embodiments, removal of water from the solution of Formula 6 increases the yield of the compound of Formula 2. In some embodiments, the yield is improved by about 1% to about 15%. In some embodiments, the yield is improved by about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 3% to about 4%, about 3% to about 5%, about 3% to about 6%, about 3% to about 7%, about 3% to about 8%, about 3% to about 9%, about 3% to about 10%, about 3% to about 15%, about 4% to about 5%, about 4% to about 6%, about 4% to about 7%, about 4% to about 8%, about 4% to about 9%, about 4% to about 10%, about 4% to about 15%, about 5% to about 6%, about 5% to about 7%, about 5% to about 8%, about 5% to about 9%, about 5% to about 10%, about 5% to about 15%, about 6% to about 7%, about 6% to about 8%, about 6% to about 9%, about 6% to about 10%, about 6% to about 15%, about 7% to about 8%, about 7% to about 9%, about 7% to about 10%, about 7% to about 15%, about 8% to about 9%, about 8% to about 10%, about 8% to about 15%, about 9% to about 10%, about 9% to about 15%, or about 10% to about 15%. In some embodiments, the yield is improved by about 1%, about 2%, about 3%, about 4%, about 5%, about 6%, about 7%, about 8%, about 9%, about 10%, or about 15%. In some embodiments, the yield is improved by at least about 1%, about 2%, about 3%, about 4%, about 5%, about 6%, about 7%, about 8%, about 9%, or about 10%. In some embodiments, the yield is improved by at most about 2%, about 3%, about 4%, about 5%, about 6%, about 7%, about 8%, about 9%, about 10%, or about 15%.

### Step IV: Preparation of a Compound of Formula 1

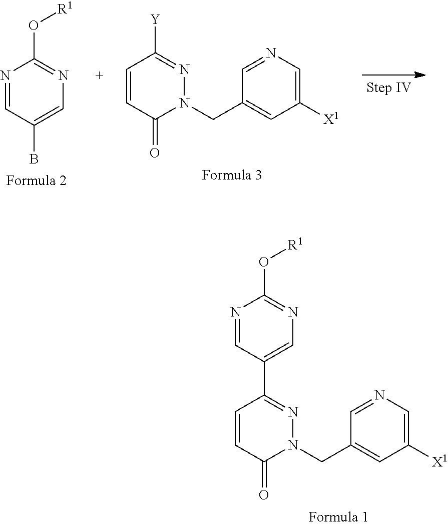

In some embodiments, a compound of Formula 2 is reacted with a compound of Formula 3 in the presence of a metal catalyst, a suitable base, in a suitable solvent, to provide a compound of Formula 1.

In some embodiments, the metal catalyst is suitable for Suzuki cross couplings. In some embodiments, the metal catalyst is a palladium catalyst. In some embodiments, the metal catalyst is selected from a palladium (0) or palladium (II) catalyst. In some embodiments, the metal catalyst comprises palladium and one or more ligand. In some embodiments, the ligand is selected from an N-heterocyclic carbene, a phosphine, a phosphite, and a bis-phosphine. In some embodiments, the ligand is selected from a phosphine, a phosphite, and a bis-phosphine. In some embodiments, the ligand is selected from a phosphine and a bis-phosphine.

In some embodiments, the phosphine is selected from trimethyl phosphine, tricyclohexylphosphine, tri-(tert-butyl)-phosphine, XantPhos, DPEPhos, XPhos, SPhos, JohnPhos, Cy-JohnPhos, Amphos, triphenylphosphine, methyldiphenylphosphine, Me4 t-BuXphos, t-BuXPhos, t-BuXantPhos, RuPhos, DavePhos, sSPhos, AdBrettPhos, BrettPhos, JackiePhos, t-BuBrettPhos, TrixiePos, t-BuDavePhos, t-BuMePhos, MePhos, PhDavePhos, VPhos, PhCPhos, XPhos-SO₃Na, water soluble SPhos, CPhos, EtCPhos, RockPhos, AlPhos, t-Bu PhCPhos, AlPhos. In some embodiments, the phosphine is selected from tricyclohexylphosphine, XantPhos, DPEPhos, XPhos, SPhos, Cy-JohnPhos, Amphos, and PhDavePhos. In some embodiments, the phosphine is Amphos,

In some embodiments, the phosphite is selected from trimethylphosphite and triphenylphosphite.

In some embodiments, the bis-phosphine is selected from bis(diphenylphosphino)methane (dppm), 1,2′-bis(diphenyl phosphino)ethane (dppe), 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf). In some embodiments, the bis-phosphine is selected from 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf).

In some embodiments, the metal catalyst is a selected from Pd(dppf)Cl₂, Pd(Amphos)₂Cl₂, Pd(dcypf)Cl₂, Pd(dtbpf)Cl₂, Pd(XantPhos)Cl₂, PdCl₂ (DPEPhos), Pd(PCy₃)Cl₂, XPhosPd G2, and RuPhos-Pd-G2. In some embodiments, the metal catalyst is Pd(Amphos)₂Cl₂.

In some embodiments, the metal catalyst is a palladacycle. In some embodiments, the metal catalyst is formed in solution.

In some embodiments, the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF. In some embodiments, the suitable base is selected from KOAc, NaHCO₃, and K₂CO₃. In some embodiments, the suitable base is K₂CO₃. In some embodiments, the suitable base is NaHCO₃.

In some embodiments, the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, methyl-t-butyl ether, water, and any combination thereof. In some embodiments, the suitable solvent is selected from dimethylformamide, N-methyl-2-pyrrolidone, tetrahydrofuran, methyl-t-butyl ether, tetrahydropyran, 1,4-dioxane, 2-methyltetrahydrofuran, water, and any combination thereof. In some embodiments, the suitable solvent is 2-methyltetrahydrofuran.

In some embodiments, B is selected from

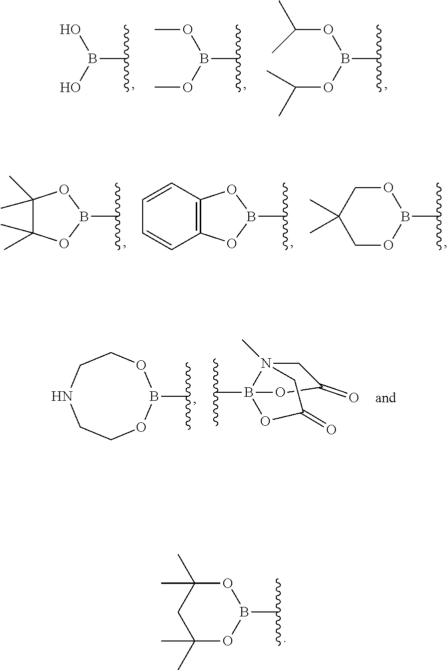

In some embodiments, B is selected from

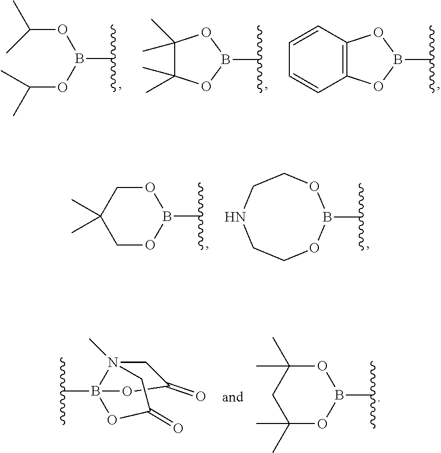

In some embodiments, B is selected from

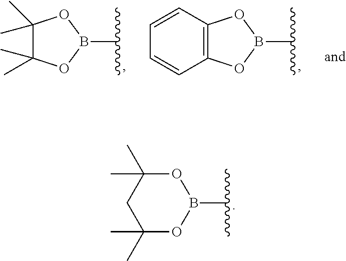

In some embodiments, B is

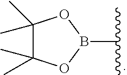 [SMILES: *B1OC(C)(C)C(C)(C)O1]

In some embodiments, B is

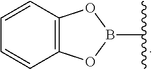 [SMILES: *B1Oc2ccccc2O1]

In some embodiments, the reaction conditions comprise a stir time of about 0.5 h to about 48 h. In some embodiments, the reaction conditions comprise a stir time of about 1 h to about 36 h. In some embodiments, the reaction conditions comprise a stir time of about 5 h to about 30 h. In some embodiments, the reaction conditions comprise a stir time of about 10 h to about 20 h.

In some embodiments, the reaction conditions comprise a reaction temperature of about 50° C. to about 120° C. In some embodiments, the reaction conditions comprise a reaction temperature of about 60° C. to about 110° C. In some embodiments, the reaction conditions comprise a reaction temperature of about 60° C. to about 90° C. In some embodiments, the reaction conditions comprise a reaction temperature of about 60° C. to about 80° C. In some embodiments, the reaction conditions comprise a reaction temperature of about 70° C. to about 80° C.

In some embodiments, the compound of Formula 1 is Compound I, the compound of Formula 2 is Compound II, and the compound of Formula 3 is Compound III. In some embodiments, the molar ratio of Compound II to Compound III is from about 1.0:1.0 to about 1.0:1.5. In some embodiments, the molar ratio of Compound II to Compound III is from about 1.0:1.0 to about 1:1.2. In some embodiments, the molar ratio of Compound II to Compound III is about 1.0:1.0. In some embodiments, the metal catalyst is Pd(Amphos)₂Cl₂. In some embodiments, the molar ratio of Compound II to the metal catalyst is between about 1.0:0.0001 to about 1.0:0.1. In some embodiments, the molar ratio of the Compound II to the metal catalyst is between about 1.0:0.001 to about 1.0:0.05. In some embodiments, the molar ratio of the Compound II to the metal catalyst is between about 1.0:0.05 to about 1.0:0.04. In some embodiments, the molar ratio of the Compound II to the metal catalyst is between about 1.0:0.01 to about 1.0:0.02. In some embodiments, the molar ratio of the Compound II to the metal catalyst is about 1.0:0.015. In some embodiments, the suitable base is NaHCO₃. In some embodiments, the molar ratio of Compound II to the suitable base is from about 1.0:5.0 to about 1.0:1.0. In some embodiments, the molar ratio of Compound II to the suitable base is from about 1.0:3.0 to about 1.0:1.0. In some embodiments, the molar ratio of Compound II to the suitable base is about 1.0:2.0.

In some embodiments, the compound of Formula 1 is recrystallized. In some embodiments, the compound of Formula 1 is recrystallized in an suitable solvent. In some embodiments, the suitable solvent is N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, methyl-t-butyl ether, water, and any combination thereof. In some embodiments, the suitable solvent is selected from dimethylformamide, N-methyl-2-pyrrolidone, tetrahydrofuran, methyl-t-butyl ether, tetrahydropyran, 1,4-dioxane, 2-methyltetrahydrofuran, water, and any combination thereof. In some embodiments, the suitable solvent is 2-methyltetrahydrofuran. In some embodiments, the suitable solvent is isopropanol.

### Step IIIa and Step IVa: Telescoped Preparation of a Compound of Formula 1

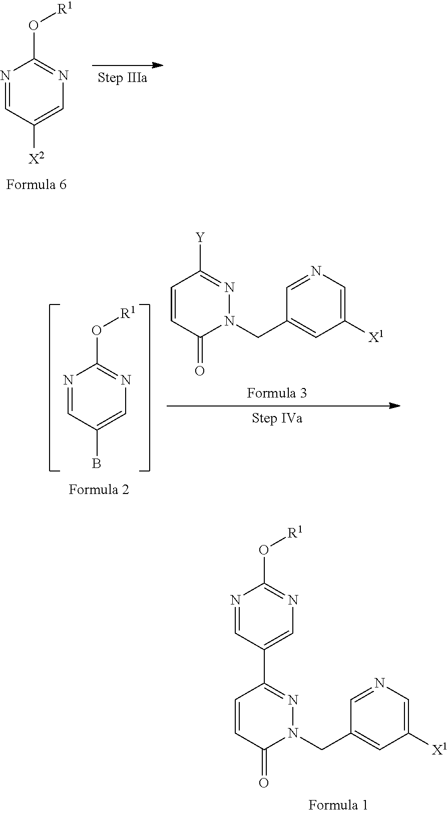

In some embodiments, a compound of Formula 6 is reacted with a boron compound in the presence of a metal catalyst, a suitable base, in a suitable solvent, to provide a compound of Formula 2 in situ. The in situ compound of Formula 2 is reacted with a compound of Formula 3 in the presence of a metal catalyst, a suitable base, in a suitable solvent, to provide a compound of Formula 1.

In some embodiments, the boron compound comprises a boron-boron bond or a boron-hydrogen bond. In some embodiments, the boron compound is selected from

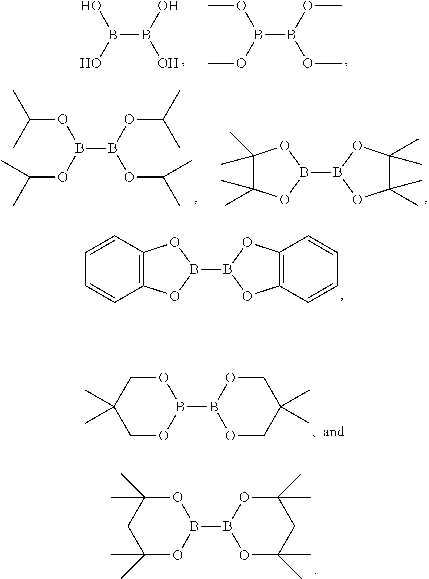

In some embodiments, the boron compound is selected from,

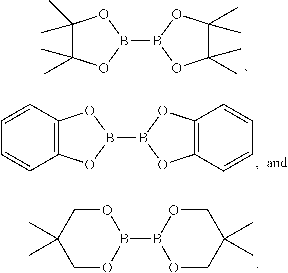

In some embodiments, the boron compound is

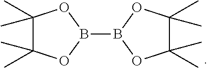

In some embodiments, the boron compound is

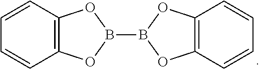 [SMILES: c1ccc2c(c1)OB(B1Oc3ccccc3O1)O2]

In some embodiments, the boron compound is

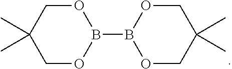

In some embodiments, the boron compound is

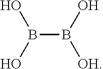

In some embodiments, B is selected from

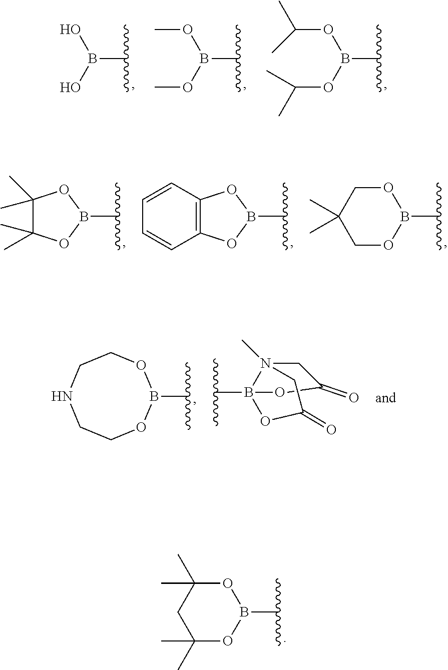

In some embodiments, B is selected from

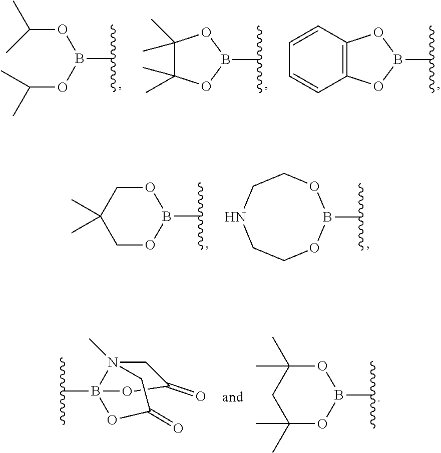

In some embodiments, B is selected from

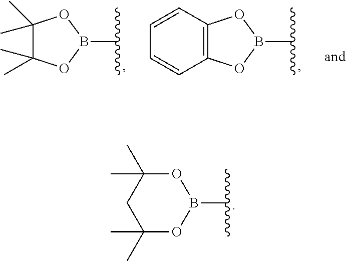

In some embodiments, B is

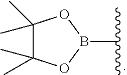 [SMILES: *B1OC(C)(C)C(C)(C)O1]

In some embodiments, B is

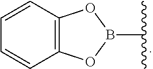 [SMILES: *B1Oc2ccccc2O1]

In some embodiments, the metal catalyst is suitable for Suzuki cross couplings. In some embodiments, the metal catalyst is a palladium catalyst. In some embodiments, the metal catalyst is selected from a palladium (0) or palladium (II) catalyst. In some embodiments, the metal catalyst comprises palladium and one or more ligand. In some embodiments, the ligand is selected from an N-heterocyclic carbene, a phosphine, a phosphite, and a bis-phosphine. In some embodiments, the ligand is selected from a phosphine, a phosphite, and a bis-phosphine. In some embodiments, the ligand is selected from a phosphine and a bis-phosphine.

In some embodiments, the phosphine is selected from trimethyl phosphine, tricyclohexylphosphine, tri-(tert-butyl)-phosphine, XantPhos, DPEPhos, XPhos, SPhos, JohnPhos, Cy-JohnPhos, Amphos, triphenylphosphine, methyldiphenylphosphine, Me4 t-BuXphos, t-BuXPhos, t-BuXantPhos, RuPhos, DavePhos, sSPhos, AdBrettPhos, BrettPhos, JackiePhos, t-BuBrettPhos, TrixiePos, t-BuDavePhos, t-BuMePhos, MePhos, PhDavePhos, VPhos, PhCPhos, XPhos-SO₃Na, water soluble SPhos, CPhos, EtCPhos, RockPhos, AlPhos, t-Bu PhCPhos, AlPhos. In some embodiments, the phosphine is selected from tricyclohexylphosphine, XantPhos, DPEPhos, XPhos, SPhos, Cy-JohnPhos, Amphos, and PhDavePhos. In some embodiments, the phosphine is Amphos,

In some embodiments, the phosphite is selected from trimethylphosphite and triphenylphosphite.

In some embodiments, the bis-phosphine is selected from bis(diphenylphosphino)methane (dppm), 1,2′-bis(diphenyl phosphino)ethane (dppe), 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf). In some embodiments, the bis-phosphine is selected from 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf).

In some embodiments, the metal catalyst is a selected from Pd(dppf)Cl₂, Pd(Amphos)₂Cl₂, Pd(dcypf)Cl₂, Pd(dtbpf)Cl₂, Pd(XantPhos)Cl₂, PdCl₂ (DPEPhos), Pd(PCy₃)Cl₂, XPhosPd G2, and RuPhos-Pd-G2. In some embodiments, the metal catalyst is Pd(Amphos)₂Cl₂. In some embodiments, the metal catalyst is Pd(dppf)Cl₂.

In some embodiments, the metal catalyst is a palladacycle. In some embodiments, the metal catalyst is formed in solution.

In some embodiments, the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF. In some embodiments, the suitable base is selected from KOAc, NaHCO₃, and K₂CO₃. In some embodiments, the suitable base is K₂CO₃. In some embodiments, the suitable base is NaHCO₃. In some embodiments, the suitable base is KOAc.

In some embodiments, the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, methyl-t-butyl ether, water, and any combination thereof. In some embodiments, the suitable solvent is selected from dimethylformamide, N-methyl-2-pyrrolidone, tetrahydrofuran, methyl-t-butyl ether, tetrahydropyran, 1,4-dioxane, 2-methyltetrahydrofuran, water, and any combination thereof. In some embodiments, the suitable solvent is 2-methyltetrahydrofuran.

In some embodiments, the reaction conditions comprise a stir time of about 0.5 h to about 48 h. In some embodiments, the reaction conditions comprise a stir time of about 1 h to about 36 h. In some embodiments, the reaction conditions comprise a stir time of about 5 h to about 30 h. In some embodiments, the reaction conditions comprise a stir time of about 10 h to about 20 h.

In some embodiments, the reaction conditions comprise a reaction temperature of about 50° C. to about 120° C. In some embodiments, the reaction conditions comprise a reaction temperature of about 60° C. to about 110° C. In some embodiments, the reaction conditions comprise a reaction temperature of about 60° C. to about 90° C. In some embodiments, the reaction conditions comprise a reaction temperature of about 60° C. to about 80° C. In some embodiments, the reaction conditions comprise a reaction temperature of about 70° C. to about 80° C.

In some embodiments, the compound of Formula 2 is Compound II, the compound of Formula 6 is Compound VI, and the boron compound is

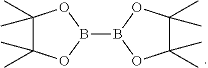

In some embodiments, the molar ratio of Compound VI to the boron compound is from about 1.0:1.0 to about 1.0:1.5. In some embodiments, the molar ratio of Compound VI to the boron compound is from about 1.0:1.0 to about 1:1.2. In some embodiments, the molar ratio of Compound VI to the boron compound is about 1.0:1.0. In some embodiments, the suitable base is KOAc. In some embodiments, the molar ratio of Compound VI to the suitable base is from about 1.0:5.0 to about 1.0:1.0. In some embodiments, the molar ratio of Compound VI to the suitable base is from about 1.0:4.0 to about 1.0:2.0. In some embodiments, the molar ratio of Compound VI to the suitable base is about 1.0:3.0. In some embodiments, the metal catalyst is Pd(dppf)₂Cl₂. In some embodiments, the molar ratio of Compound VI to the metal catalyst is between about 1.0:0.0001 to about 1.0:0.1. In some embodiments, the molar ratio of the Compound VI to the metal catalyst is between about 1.0:0.001 to about 1.0:0.05. In some embodiments, the molar ratio of the Compound VI to the metal catalyst is between about 1.0:0.05 to about 1.0:0.04. In some embodiments, the molar ratio of the Compound VI to the metal catalyst is between about 1.0:0.01 to about 1.0:0.02. In some embodiments, the molar ratio of the Compound VI to the metal catalyst is about 1.0:0.015.

In some embodiments, the compound of Formula 1 is Compound I, the compound of Formula 2 is Compound II, and the compound of Formula 3 is Compound III. In some embodiments, the molar ratio of Compound II to Compound III is from about 1.0:1.0 to about 1.0:1.5. In some embodiments, the molar ratio of Compound II to Compound III is from about 1.0:1.0 to about 1.0:1.2. In some embodiments, the molar ratio of Compound II to Compound III is about 1.0:1.0. In some embodiments, the metal catalyst is Pd(Amphos)₂Cl₂. In some embodiments, the molar ratio of Compound II to the metal catalyst is between about 1.0:0.0001 to about 1.0:0.1. In some embodiments, the molar ratio of the Compound II to the metal catalyst is between about 1.0:0.001 to about 1.0:0.05. In some embodiments, the molar ratio of the Compound II to the metal catalyst is between about 1.0:0.05 to about 1.0:0.04. In some embodiments, the molar ratio of the Compound II to the metal catalyst is between about 1.0:0.01 to about 1.0:0.02. In some embodiments, the molar ratio of the Compound II to the metal catalyst is about 1.0:0.015. In some embodiments, the suitable base is NaHCO₃. In some embodiments, the molar ratio of Compound II to the suitable base is from about 1.0:5.0 to about 1.0:1.0. In some embodiments, the molar ratio of Compound II to the suitable base is from about 1.0:3.0 to about 1.0:1.0. In some embodiments, the molar ratio of Compound II to the suitable base is about 1.0:2.0.

In some embodiments, compounds described herein are synthesized as outlined in the Examples.

### Compounds

Disclosed herein, in some embodiments, is a compound represented by Formula 9:

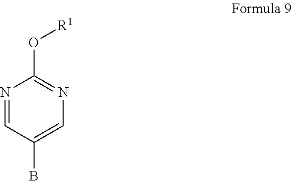

or a salt thereof, wherein:
R¹ is —CF₃, —CHF₂, —CH₂CF₃, and —CH₂CHF₂; and
B is selected from a boronic acid and a boronic ester.

In some embodiments, R¹ is —CH₂CF₃. In some embodiments, B is selected from

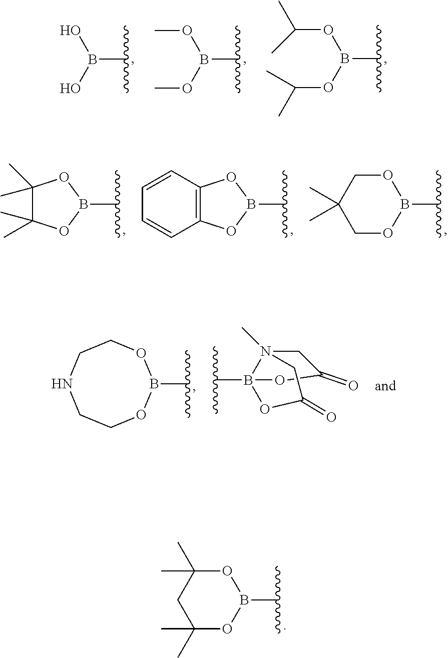

In some embodiments, B is selected from

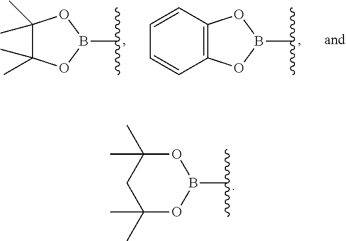

In some embodiments, the compound is Compound II depicted below:

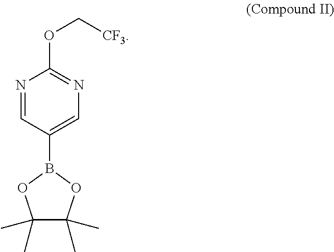 [SMILES: CC1(C)OB(c2cnc(OCC(F)(F)F)nc2)OC1(C)C]

### MIDA Boronates

In some embodiments, methyliminodiacetic acid (MIDA) boronates are bound to the heteroaryl group prior to palladium catalyzed transmetalation. For example, Compound A below may be synthesized and utilized in place of Compound II.

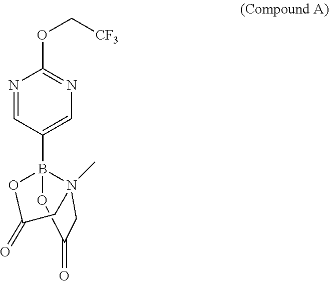

Compound A may be synthesized via the reaction of BCl₃, N-methyliminodiacetic acid and Compound VI.

### Heavy Metal Scavengers

In some embodiments, the compound of Formula 1 (such as Compound I) or the compound of Formula 2 (such as Compound II) is further treated with a metal scavenger to remove residual palladium. In some embodiments, the metal scavenger comprises SiO₂, charcoal, aqueous solution of L-cysteine, a Silicycle metal scavenger, Si-thiol, SiliaBond DMT, SiliaBond Cysteine, or 3-mercaptopropyl ethyl sulfide silica. In some embodiments, the ratio of the metal catalyst to the scavenger (w/w) is about 1:100, 1:50, 1:30, 1:25, 1:20, 1:15, 1:10, 1:5, 1:3, 1:2, or about 1:1

In some of these embodiments, palladium levels are reduced to about 10 ppm. In some of these embodiments, palladium levels are reduced sufficiently to be undetectable.

In some embodiments, the presence of residual heavy metal (e.g. palladium) impurities is determined by utilizing methods known in the art. In some embodiments, the presence of residual heavy metal (e.g. palladium) impurities is determined by the use of inductively coupled plasma mass spectrometry (ICP-MS). In some embodiments, the presence of residual heavy metal (e.g. palladium) impurities is determined by the use of techniques described in U.S. Pharmacopeia General Chapter <231> Heavy Metals.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “C_{x-y}” or “Cₓ-C_{y}” when used in conjunction with a chemical moiety, such as haloalkyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “halo” or, alternatively, “halogen” or “halide,” means fluoro, chloro, bromo or iodo. In some embodiments, halo is fluoro, chloro, or bromo.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are obtained by reacting a compound disclosed herein with an acid. In some embodiments, the compound disclosed herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts are obtained by reacting a compound disclosed herein with a base. In some embodiments, the compound disclosed herein is acidic and is reacted with a base. In such situations, an acidic proton of the compound disclosed herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, n-propanol, isopropanol, n-butanol, t-butanol, sec-butanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, tetrahydropyran, and tetrahydrofuran.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound A, comprise an organic solvent(s). In some embodiments, compositions comprising Compound A include a residual amount of an organic solvent(s). In some embodiments, compositions comprising Compound A comprise a residual amount of a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from ethyl acetate, isopropyl acetate, tert-butylmethylether, heptane, isopropanol, and ethanol.

In some embodiments, the compositions comprising Compound A include a detectable amount of an organic solvent. In some embodiments, the organic solvent is a Class 3 solvent.

In other embodiments are compositions comprising Compound A wherein the composition comprises a detectable amount of solvent that is less than about 1%, wherein the solvent is selected from acetone, 1,2-dimethoxyethane, acetonitrile, ethyl acetate, tetrahydrofuran, methanol, ethanol, heptane, and 2-propanol. In a further embodiment are compositions comprising Compound A wherein the composition comprises a detectable amount of solvent which is less than about 5000 ppm. In yet a further embodiment are compositions comprising Compound A, wherein the detectable amount of solvent is less than about 5000 ppm, less than about 4000 ppm, less than about 3000 ppm, less than about 2000 ppm, less than about 1000 ppm, less than about 500 ppm, or less than about 100 ppm.

### EXAMPLES

The following illustrative examples are representative of embodiments of the methods described herein and are not meant to be limiting in any way.

### Example 1—Synthesis of 2-((5-fluoropyridin-3-yl)methyl)-6-(2-(2,2,2-trifluoroethoxy)pyrimidin-5-yl)pyridazin-3 (2H)-one (Compound I)

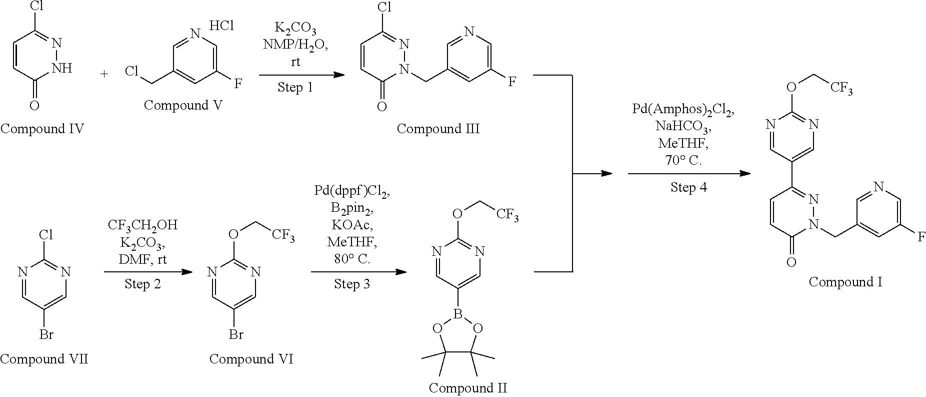

### Step 1. Synthesis of 6-chloro-2-((5-fluoropyridin-3-yl)methyl)pyridazin-3 (2H)-one (Compound III)

Preparation of Compound III was completed using 0.570 kg (4.38 mol) of Compound IV and Compound V (0.788 kg, 4.32 mol, 0.99 equiv.), and K₂CO₃ (1.81 kg, 13.1 mol, 3 equiv.) in NMP/water. The reaction temperature rose from 20° C. to 34° C. during the addition of water and off-gassing (CO₂ evolution) was observed during the K₂CO₃ addition, which was added over 10 min. HPLC analysis after 15 h revealed <1 HPLC A % of Compound IV. (PDF). After the inorganics were filtered from the reactor, the cake was washed with IPAc (10 vol). Filtrates and washes were combined and washed with aq. LiCl and layers were separated. The aqueous layer was back-extracted with IPAc (2 vol). The organic layers were combined and washed with water. The layer separations were rapid, requiring <10 minutes. The final IP Ac layer containing product was filtered using a 0.45-micron filter cartridge and then concentrated to dryness on a Büchi rotary evaporator. The resulting semi-solid was treated with deionized water (10 vol) in the Büchi bulb and agitated by rotating the distillation bulb under atmospheric pressure until a uniform slurry was obtained and filtered. The filtered solids were dried on the filter funnel for several hours under a nitrogen tent by applying vacuum and then dried in a vacuum oven at 35° C. until constant weight was achieved. After drying, a total of 817.5 g of Compound III was isolated as a tan solid, 78% yield. The isolated Compound III was found to be 97.8 A % pure by HPLC and found to be 99 wt. % pure by quantitative NMR analysis. The isolated Compound III was found to be free of Compound V.

### Step 2. Synthesis of 5-bromo-2-(2,2,2-trifluoroethoxy)pyrimidine (Compound VI)

Synthesis of Compound VI was performed using 20 g Compound VII, 1.6 equiv. K₂CO₃, and 1.1 equiv. 2,2,2-trifluoroethanol at 19° C. in DMF. The reaction required stirring overnight (>18 h) to afford >99% HPLC A % Compound VI. Upon completion of the reaction, addition of cold H₂O to the reaction did not dissolve all salts. Ethyl Acetate was added to the mixture and, when transferred to a separatory funnel, a tri-layer formed. Eventually, Compound VI was isolated after concentration. An additional round of aqueous washes for this material was necessary to remove excess DMF. The material was azeotropically dried with PhMe. 22.3 g of Compound VI was obtained (98.8% isolated yield). Scaling up of the procedure utilizing 5.945 kg Compound VII, 6.90 kg K₂CO₃, and 3.4 kg of 2,2,2-trifluoroethanol afforded 8.45 kg of Compound VI (97.5% isolated yield).

### Step 3. Synthesis of 5-(4,4,5,5-tetramethyl-1,3,2-dioxaborolan-2-yl)-2-(2,2,2-trifluoroethoxy)pyrimidine (Compound II)

A 50 L reactor was charged with Compound VI (1.80 kg, 7.00 mol, 1 equiv.), B₂Pin₂ (1.79 kg, 7.01 mol, 1 equiv.), KOAc (2.06 kg, 20.99 mol, 3 equiv.) and 10.8 L of 2-MeTHF under N₂. The mixture was stirred for 1 hour under N₂. Metal catalyst Pd(dppf)Cl₂ (77 g, 0.11 mol, 0.015 equiv.) was added to the reactor. The reactor was heated to 80-85° C. and progress of the reaction was monitored by HPLC. Upon completion, H₂O was added. The organic layer was washed, filtered, and evaporated. The crude product was recrystallized from heptane to afford 1.2 kg of Compound II in 55% yield. The isolated Compound II was found to be 99 A % by HPLC analysis and 94 wt. % by QNMR analysis.

Prior to recrystallization from heptane, the crude product was dissolved in MTBE and filtered through a silica gel pad. The MTBE was removed and replaced with heptane to form a suspension, filtered, and chilled to yield crystalline Compound II. Compound II was isolated by filtration and washed with cold heptane (≤0° C.) and dried.

A second crop was isolated from the mother liquor following concentration, filtration, and washing with cold heptane to yield an additional 0.21 kg in 6.6% yield. The isolated compound II was found to be >99 A % by HPLC analysis and 65 wt. % by QNMR analysis.

### Step 4. Synthesis of 2-((5-fluoropyridin-3-yl)methyl)-6-(2-(2,2,2-trifluoroethoxy)pyrimidin-5-yl)pyridazin-3 (2H)-one (Compound I)

A 50 L reactor was charged with Compound II (1186.6 g, 95 wt. %, 1.0 eq.), Compound III (888.4 g, 1.0 eq.) and degassed 2-MeTHF (11.0 L) under N₂. After a few minutes of stirring, degassed water (5.9 L) and NaHCO₃ (622.9 g, 2.0 eq.) was added, followed by the addition of Pd (Amphos) 2Cl₂ (39.37 g, 0.015 eq.). The contents of the 50 L reactor were heated to 70° C. and a gentle reflux was observed. The reaction mixture was held at 70° C. for 3 h and monitored by HPLC. The reaction was cooled to room temperature, the aqueous layer discarded and the organic layer reduced.

Purification 1: The solid was dissolved in EtOAc and filtered through a silica gel plug. The solution was concentrated to a slurry and 4 L of 2-propanol added. The solution was heated until homogenous and subsequently cooled, resulting in a slurry. The solid collected via filtration to afford 1126 g of Compound I (isolated yield 79.6%). Compound I was found to be 99.36% HPLC A % for product by HPLC.

Alternate Purification 2: The solid was dissolved in EtOAc and treated with functionalized silica gel and DARCO activated carbon. The functionalized silica gel and DARCO activated carbon were removed by filtration. The solution was concentrated to a slurry and 4 L of 2-propanol added. The solution was heated until homogenous and subsequently cooled, resulting in a slurry. The solid collected via filtration to afford 1126 g of Compound I (isolated yield 79.6%). Compound I was found to be 99.36% HPLC A % for product by HPLC.

### Example 2—Alternative Synthesis of 2-((5-fluoropyridin-3-yl)methyl)-6-(2-(2,2,2-trifluoroethoxy)pyrimidin-5-yl)pyridazin-3 (2H)-one (Compound I)

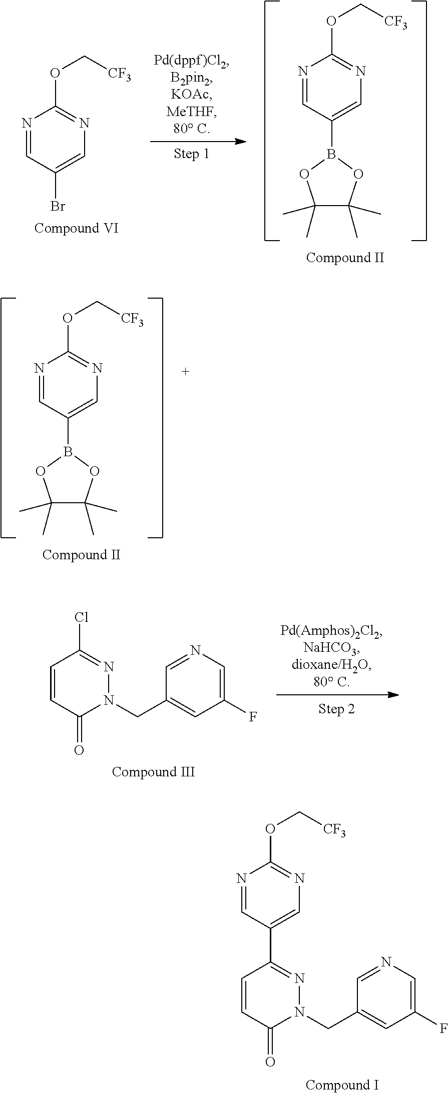

### Step 1. In situ generation of 5-(4,4,5,5-tetramethyl-1,3,2-dioxaborolan-2-yl)-2-(2,2,2-trifluoroethoxy)pyrimidine (Compound II)

A telescoped procedure for the generation of Compound I was developed. On a small scale, 1 g of Compound VI was treated with B₂pin₂ (1 eq) in the presence of Pd(dppf)Cl₂ (1.5 mol %) and KOAc (3 eq) in 2-MeTHF at 80° C. The borylation reaction was complete in 3.5 h.

### Step 2. Synthesis of 2-((5-fluoropyridin-3-yl)methyl)-6-(2-(2,2,2-trifluoroethoxy)pyrimidin-5-yl)pyridazin-3 (2H)-one (Compound I)

After concentration, the reaction mixture containing Compound II was subjected to Suzuki coupling with Compound III (0.9 eq) in dioxane in the presence of 1.0 mol % Pd(Amphos)₂Cl₂, NaHCO₃ (3 eq) and water (5 V) at 80° C. for 19 h resulting in a final reaction mixture with 92.5% LCAP for the product Compound I and 3.6% LCAP for the homocoupling product of Compound VI, 2,2′-bis(2,2,2-trifluoroethoxy)-5,5′-bipyrimidine. After the reaction was worked up, the product was isolated from 2-propanol (1.08 g, 73% yield). HPLC showed 99.8% LCAP for the product. After 10% methyl thiourea silica gel treatment to remove palladium a total 0.7 g (47% yield) of Compound I was obtained which contained 31 ppm Pd.

Scaling up, a 20 g scale up telescoped process using Pd(dppf)Cl₂/2-MeTHF for the borylation step and Pd(Amphos)₂Cl₂/1,4-Dioxane for the Suzuki reaction step encountered a significantly slower rate (23 h) for the borylation step.

Purification 1: The reaction was worked up as usual and after crystallization step the isolated Compound I (14.3 g) was found to be only 92.3% LCAP with 6.0% an impurity resulting from the homo coupling of Compound III, 1,1′-bis((5-fluoropyridin-3-yl)methyl)-[3,3′-bipyridazine]-6,6′(1H,1′H)-dione. The overall yield was 54%.

### Example 3—Synthesis of 2-((5-fluoropyridin-3-yl)methyl)-6-(2-(2,2,2-trifluoroethoxy)pyrimidin-5-yl)pyridazin-3 (2H)-one (Compound I)

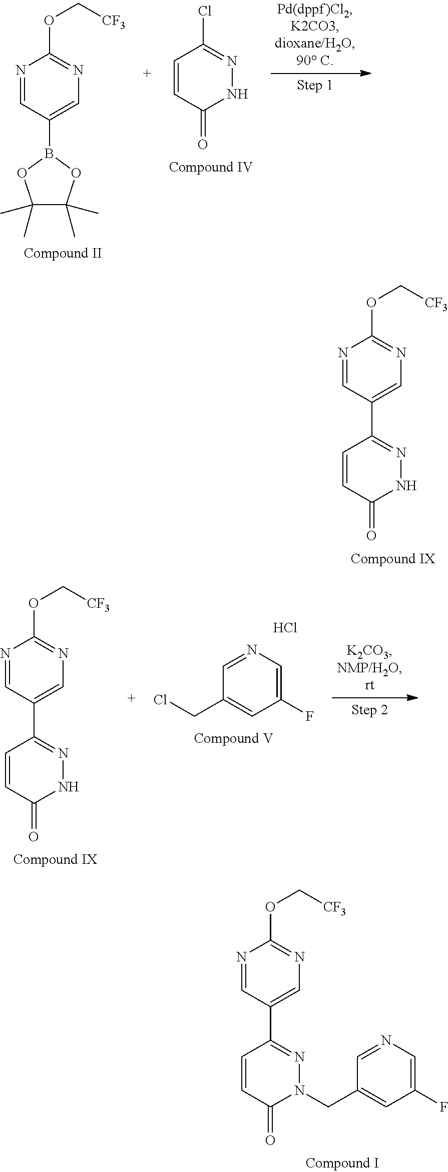

### Step 1. Synthesis of 6-[2-(2,2,2-trifluoroethoxy)pyrimidin-5-yl]-2,3-dihydropyridazin-3-one (Compound IX)

To a mixture of Compound II (4.2 g, 18.93 mmol, 1.0 equiv) in dioxane (40 mL) were added Compound IV (3.31 g, 18.916 mmol, 1.00 equiv), Pd(dppf)Cl₂ (0.69 g, 0.943 mmol, 0.05 equiv), K₂CO₃ (3.92 g, 28.387 mmol, 1.5 equiv) and H₂O (4 mL). The flask was purged and maintained with an inert atmosphere of nitrogen. The resulting solution was stirred for 2 h at 90° C. The solution was diluted with water and extracted with EtOAc (30 mL×3). The combined organics were washed with brine, dried over Na₂SO₄ and the solvent removed in vacuo. Purification by chromatography on silica gel (Flash 300 g, 50-100% EtOAc: cyclohexane) afforded Compound IX as a brown solid (3.0 g, 58.24%). LC/MS (ESI): 273 [M+H]⁺. Scaling up of the procedure to kilogram scales led to decreased yields. Further, Compound IX was found to have limited solubility, as seen in Table 1 below.

TABLE 1

| Entry | Solvent | mg/mL |
|---|---|---|
| 1 | DCE | 0.15 |
| 2 | Acetone | 1.59 |
| 3 | thietane 1,1-dioxide | 6.35 |
| 4 | DME | 3.26 |
| 5 | THF | 2.73 |
| 6 | ACN | 1.5 |
| 7 | toluene | 0 |
| 8 | 1,4-dioxane | 2.9 |
| 9 | N-heptane | 0 |
| 10 | DMSO | 45 |
| 11 | MeOH | 2.5 |
| 12 | EtOH | 1.0 |
| 13 | i-PrOH | 0.5 |
| 14 | t-BuOH | 0.1 |
| 15 | MTBE | 0.1 |
| 16 | EA | 0.7 |
| 17 | DCM | 0.3 |
| 18 | DMF | 21 |
| 19 | DMA | 13 |
| 20 | NMP | 44 |
| 21 | DME | 10 |

### Step 2. Synthesis of 2-((5-fluoropyridin-3-yl)methyl)-6-(2-(2,2,2-trifluoroethoxy)pyrimidin-5-yl)pyridazin-3 (2H)-one (Compound I)

Compound IX (0.736 kg, 4.04 mol) was charged to a jacketed 10 L reactor, followed by NMP (4 L). Jacket temperature of the reactor was set to 15° C. Stirring was started and DI water (1 L) was charged. An exotherm to about 26° C. was observed. Potassium carbonate (1.523 kg, 11 mol, 3.01 eq.) was charged in portions such that the temperature did not rise above 25° C. Compound V (1.0 kg) was added using a funnel in one portion. The resulting mixture was allowed to stir at 23±2° C. for 15-20 min after which an additional 1 L of 1:4 (v/v) Water: NMP was charged to aid the stirring. The reaction mixtures was continued to stir at 23±2° C. and progress of the reaction was monitored by HPLC. After the reaction was judged complete, the Jacket temperature of the reactor was set at 10° C. and 5 L water was added which resulted in a mild exotherm. the reaction mixture was drained and poured into 90 L of water in a 100 L reactor. The mixture was allowed to stir at room temperature for 60 minutes, after which solids were filtered through a medium-frit sintered glass filter-funnel. The off-white cake was washed with 6.5 L of water. Using the same procedure, two more runs were completed using 1.0 and 0.695 kg of Compound IX, respectively. The solid was recrystallized from EtOH to afford 3.35 kg of Compound I in an 88% yield. The isolated material was found to be >99.9 A % by the HPLC.

### Example 4—Alternative Synthesis of 2-((5-fluoropyridin-3-yl)methyl)-6-(2-(2,2,2 trifluoroethoxy)pyrimidin-5-yl)pyridazin-3 (2H)-one (Compound I)

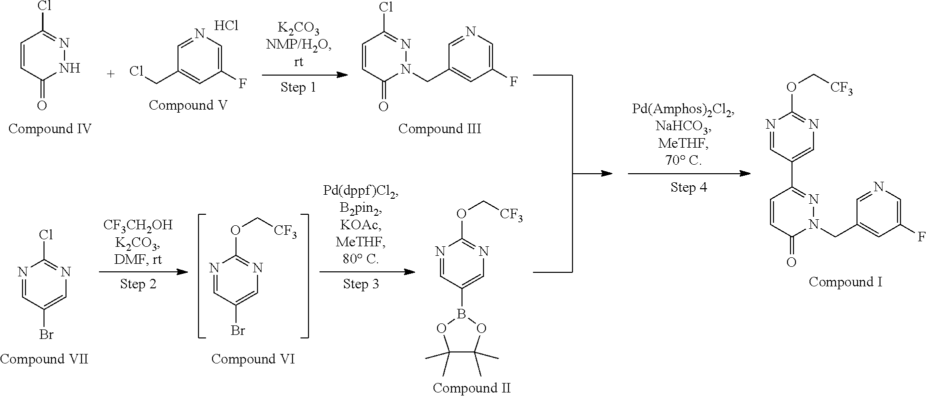

### Step 1. Synthesis of 6-chloro-2-((5-fluoropyridin-3-yl)methyl)pyridazin-3 (2H)-one (Compound III)

To a 12 L 3-neck rbf equipped with nitrogen inlet, mechanical stirrer and internal thermocouple was charged H₂O (1.60 kg) and K₂CO₃ (325 mesh; 0.740 kg). The solution was diluted with NMP (0.80 kg) and Compound V (0.322 kg) was charged to the stirring solution in 4 roughly equal portions every 5 minutes. After all the solids were dissolved a solution of Compound IV in NMP (0.235 kg in 0.850 kg NMP) was charged to the mixture over the course of 1 hour, while maintaining the reaction temperature ≤40° C. The mixture was cooled to 25° C. and stirred near this temperature for 18.5 hours. IPC analysis by HPLC indicated <1% Compound V remaining. After this time, the reaction mixture was diluted with H₂O (6.40 kg) and the resultant tan/orange slurry adjusted to 6-10° C. before stirring for 3 hours. The reaction mixture was filtered on a Buchner filtration funnel and the solids washed with water (2×2 kg) when pH of the filtrate=7.5-7.0. The solids were dried on the funnel for 30 minutes before transferring to a vacuum-drying oven. Solids were dried to a constant weight (<1% difference between subsequent weighings) over 21 hours at 50° C. Isolated 0.314 kg of a tan solid of Compound III (74.1% yield; 100% AUC).

### Step 2. Synthesis of 5-bromo-2-(2,2,2-trifluoroethoxy)pyrimidine (Compound VI)

507 g of Compound VII (2.62 mol, lot 73197) was charged to the reaction flask, followed by N,Ndimethylformamide (3.28 L). The reactor contents were agitated at 300 rpm while charging 2,2,2-trifluoroethanol (lot 72775) as a neat liquid (2.88 mol, 1.1 eq) in one portion. K₂CO₃ (325 mesh, 4.19 mol, 1.6 eq) was charged as a solid in portions over 10 minutes. The reaction mixture was cooled naturally to 30° C. and stirred overnight (18 hour stir time) when another aliquot of the reaction mixture diluted 1/40 in 50:50 MeCN/H₂O for HPLC analysis. The reaction mixture was filtered through 7 μm polypropylene filter cloth and the salts washed with 2-MeTHF (2.5 kg). The washes were combined with the filtrate and mixture was diluted with 10% aqueous LiCl (2.5 kg) and the resultant phases stirred for 15 minutes at 25-30° C. Agitation was ceased and the layers allowed to split. After recommencing a low agitation speed (<20 rpm) and stirring for an additional 20 minutes the layers settled with desired product in the bottom layer. The layers were then separated and the top aqueous layer was charged with additional 2-MeTHF for extraction (2.5 kg). The combined organic layers were sequentially washed with USP water (2×2.5 kg) and 10% aqueous LiCl (2.5 kg). In each case, the bottom layer was removed with organic product in the top layer. The washed organic layer was azeotroped for remaining water concentrated under vacuum to ˜1.2 L (bath temperature 35-45° C.) and an additional 2.5 kg of 2-MeTHF. An aliquot of this solution was submitted for Karl Fischer analysis (Result: 0.4% H₂O content. Target: ≤1%). The solution was then collected and massed at 3.80 kg. An HPLC assay determined 17.1% w/w Compound VI (0.65 kg in solution; 96.4% isolated yield;). 99.7% AUC. The solution was carried forward as-is into Step 3.

### Step 3. Synthesis of 5-(4,4,5,5-tetramethyl-1,3,2-dioxaborolan-2-yl)-2-(2,2,2-trifluoroethoxy)pyrimidine (Compound II)

To a 12 L 3-neck rbf equipped w/mechanical stirrer, N₂ inlet, internal thermocouple and mechanical stirrer was charged 3.8 kg of Compound VI solution (17.3% w/w in 2-MeTHF; 0.65 kg; 2.53 mol). The solution was agitated and B₂pin₂ (0.71 kg, 2.79 mol) was charged as a solid in one portion. To this mixture, KOAc (0.75 kg, 7.68 mol) was charged and the vessel contents were subsurface sparged with N₂ for 10 minutes, Pd(dppf)Cl₂ catalyst (0.028 kg, 0.038 mol) was added as a solid under nitrogen and the vessel contents heated to 70-75° C. for 22 hours via external heating mantle. The mixture was cooled to 40° C. and USP water (2 kg) was added. The mixture was stirred for 25 minutes. After this time, the aqueous (bottom) layer was removed and the organic layer washed twice w/5% aqueous NaCl (3.25 kg per wash). The organic layer was diluted with n-heptane (6.2 kg) and stirred for 30 minutes. To this suspension was charged Silica (Siliaflash™ (Silicycle), 60-200 μm size, 0.65 kg) and the slurry stirred for 20 minutes. This suspension was filtered through a Celite pad (0.2 kg). The solids on the pad were washed with 3:1 v/v n-heptane: 2-MeTHF (1.4 kg) and the wash combined with the filtrate. The combined filtrate and washes were distilled under a partial vacuum (external heating temperature 40-45° C.) to a concentrate of ˜2 L. To this concentrate, n-heptane (4.5 kg) was charged and distillation repeated down to ˜2 L. This concentrate was again diluted with n-heptane (1.8 kg). 1H NMR analysis showed no remaining 2-MeTHF in the solution. The slurry was chilled to 0-5° C. while stirring. Once at this temperature a small amount of 2-MeTHF (0.08 kg) was added and the slurry aged at temperature for 2 hours. After this time, the slurry was filtered on a 10″ Buchner funnel (7 μm polypropylene filter cloth). The mother liquor was looped back into the original vessel to collect any residual solid and deposit onto the filter cake. The cake was vacuum-dried under nitrogen blanket for 20 minutes and the partially dried solids transferred to a vacuum-drying oven set at 40° C. After 3 days, off-white solids were removed from the drying oven and massed at 0.49 kg (63.3% isolated yield) of Compound II (94.7% AUC).

Alternative Route: to a 20 L jacketed reaction vessel equipped w/22 mm glass stir shaft (PTFE blades), mechanical stirrer, nitrogen inlet, pressure-equalizing addition funnel, Dean-stark adapter w/reflux condenser (vented to mineral oil bubbler) and thermocouple was sequentially charged B₂Pin₂ (0.971 kg), 2-MeTHF (6.60 kg), KOAc (0.982 kg) The mixture was agitated (160 rpm) and heated to reflux (approximately 80° C.). Approximately 1.4 kg of 2-MeTHF distillate was collected and drained via the Dean-Stark trap over the course of 3 hours. After distillation was complete Pd(dppf)Cl₂ (0.036 kg) was added as a solid in one portion under a blanket of nitrogen and Compound VI solution was transferred to the addition funnel and added in portions over the course of 45 minutes. The reaction was heated at 75-80° C. and sampled for reaction completion after 16 hours. A kicker charge of B₂pin₂ (90 g), KOAc (90 g) and Pd(dppf)Cl₂ (3.7 g) was added and the reaction resampled after an additional 4 hours of reaction time. Following the completion of the reaction, the crude product was worked up as described above. After 3 days, off-white solids were removed from the drying oven and massed at 0.674 kg (66.3% isolated yield) of Compound II (99.9% AUC).

Azeotropic removal of water prior to the initiation of the Miyaura borylation to form Compound II followed by a controlled addition of Compound VI (41% w/w, H₂O<0.1% w/w) to the heating mixture was successful in minimizing the formation of undesired side products, such as the homocoupled impurity of Compound VI (below). Azeotroping under nitrogen also served as a means of degassing the mixture prior to the reaction start. The reduction in homocoupled impurity and solvent degassing led to a 3.0% isolated yield increase and a 5.2% purity increase as determined by HPLC in the procedures described above. The amount of homocoupled impurity is typically reduced to less than 1% (0.07% in the procedure above) when the water is azeotropically removed. Failing to remove the water typically results in 4-7% homocoupled impurity, however values of up to 45% have been observed during development of the manufacturing procedures. Therefore, azeotropic removal of water serves to reduce yield variability in the manufacturing procedure.

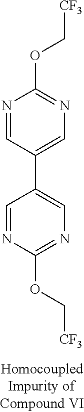 [SMILES: FC(F)(F)COc1ncc(-c2cnc(OCC(F)(F)F)nc2)cn1]

### Step 4. Synthesis of 2-((5-fluoropyridin-3-yl)methyl)-6-(2-(2,2,2-trifluoroethoxy)pyrimidin-5-yl)pyridazin-3 (2H)-one (Compound I)

To a 12 L 3-neck rbf equipped with mechanical stirrer (glass rod w/PTFE stir blade), nitrogen inlet, internal thermocouple and external heating mantle was charged Compound III (0.30 kg), 2-MeTHF (3.3 kg) and Compound II (0.41 kg). The reactor contents were set to stir and NaHCO₃ (0.21 kg) was added followed by H₂O (2.0 kg). The mixture was sub-surface sparged with nitrogen for 10 minutes before charging Pd(amphos)₂Cl₂ (0.013 kg) as a solid in one portion under a blanket of nitrogen. The sparging needle was removed and the mixture heated to 70-80° C. under a gentle nitrogen blanket. The mixture was vigorously stirred at temperature for 4 hours when IPC analysis indicated 0.1% Compound III remaining (target specification ≤1% relative to Compound I). The mixture was cooled to room temperature with overnight stirring for 13 hours.

Stirring was ceased and the layers allowed to settle. The aqueous (bottom) layer was removed and the organic layer concentrated under vacuum to ˜2.2 L. Ethyl acetate (3.6 kg) was charged to the vessel and the concentration was repeated once more to remove ˜4.5 L of distillate. An additional 3.7 kg of EtOAc was charged. The organic layer was washed with aqueous 5% (w/w) brine (2.0 kg) and then to the organic layer was charged silica-supported thiourea metal scavenger (Carbosynth, 0.066 kg) and the mixture stirred at room temperature under nitrogen for 24 hours. At this time, the stirring reaction mixture was charged with additional silica (Siliaflash®, 60-200 μm 60 Å, 0.48 kg) and stirred for 30 minutes. The mixture was then filtered through a Buchner filter funnel and the solids washed twice with EtOAc (1.4 kg per wash). The filtrates and washes were cartridge-filtered under vacuum to a 20 L rotovap receiving flask. The external heating bath was set to 42-45° C. and EtOAc was removed to an approximate volume of ˜1.5 L.

Isopropanol (0.98 kg) was charged to the bulk mixture and concentration under vacuum continued until an approximate volume of 1.5 L was reached. Two additional chase-distillation cycles were completed (0.92 kg isopropanol added per chase) and an approximate volume of 1.5 L was reached. The bulk stream was diluted with additional isopropanol (0.92 kg) and the mixture transferred to a 5 L 3-neck rbf equipped with mechanical stirrer, nitrogen inlet and internal thermocouple. The mixture was heated to 60±5° C. via external heating mantle and stirred at this temperature until complete dissolution of the solids was noted. The solution was then allowed to cool to 50-55° C. The batch was seeded with milled Compound I (3.3 g, 1.1% w/w relative to Compound III input) and held at this temperature for 1 hour. The heat source was turned off and the reaction mixture was cooled to ambient temperature and stirred for 16 hours. At this time, the concentration of Compound I in the supernatant was assessed by HPLC to be 24.2 mg/mL. The bulk slurry was filtered on a 7-micron polypropylene filter cloth under vacuum and the filter cake washed with cold IPA (2×0.32 kg, held at 3-5° C.). The washed solids were dried under vacuum with a nitrogen blanket for 45 minutes. The bulk solids were then transferred to a vacuum drying oven with a nitrogen bleed and dried at 45° C. for 2 days. White solids were massed at 382.8 g of Compound I (80.2% yield; 99.7% AUC5).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A process for the preparation of a compound of Formula 1: 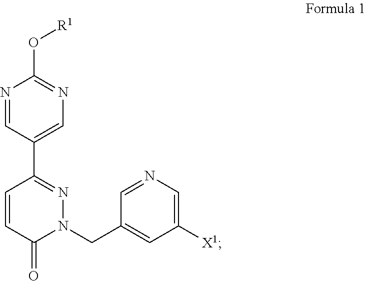
wherein X¹ is a halogen; and
R¹ is C₁-C₆ haloalkyl;
comprising:
reacting a compound of Formula 2: 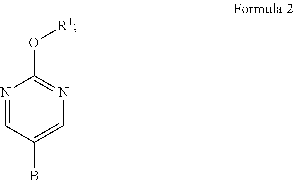
wherein R¹ is C₁-C₆ haloalkyl; and
B is selected from a boronic acid and a boronic ester;
with a compound of Formula 3 under coupling conditions: 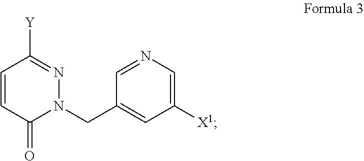
wherein X¹ is a halogen; and
Y is a leaving group;
in the presence of a metal catalyst, a base, in a suitable solvent, to provide a compound of Formula 1.

2. The process of claim 1, wherein R¹ is C₁-C₃ haloalkyl.

3. The process of claim 2, wherein R¹ is selected from —CF₃, —CHF₂, —CH₂CF₃, and —CH₂CHF₂.

4. The process of any one of claims 1 to 3, wherein B is selected from

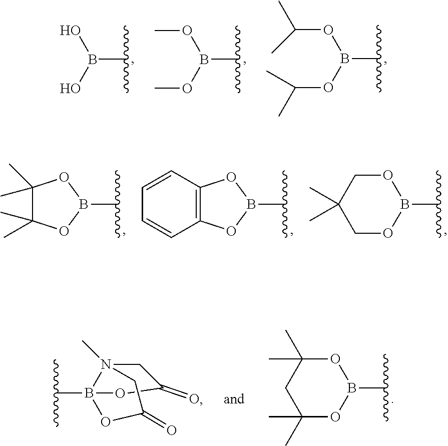

5. The process of claim 4, wherein B is selected from

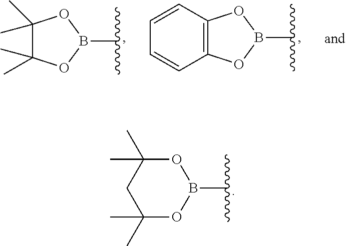

6. The process of any one of claims 1 to 5, wherein Y is selected from halogen and pseudo-halide.

7. The process of claim 6, wherein Y is selected from halogen and -OTf.

8. The process of claim 7, wherein Y is selected from —Cl and —Br.

9. The process of any one of claims 1 to 8, wherein X¹ is selected from —F and —Cl.

10. The process of any one of claims 1 to 9, wherein the metal catalyst is a palladium catalyst.

11. The process of any one of claims 1 to 10, wherein the metal catalyst is selected from a palladium (0) or palladium (II) catalyst.

12. The process of any one of claims 1 to 11, wherein the metal catalyst comprises palladium and one or more ligand, wherein the one or more ligand is selected form a phosphine, phosphite, a bis-phosphine, and an N-heterocyclic carbene.

13. The process of claim 12, wherein the phosphine is selected from trimethyl phosphine, tricyclohexylphosphine, tri-(tert-butyl)-phosphine, XantPhos, DPEPhos, XPhos, SPhos, JohnPhos, Cy-JohnPhos, Amphos, triphenylphosphine, methyldiphenylphosphine, Me4 t-BuXphos, t-BuXPhos, t-BuXantPhos, RuPhos, DavePhos, sSPhos, AdBrettPhos, BrettPhos, JackiePhos, t-BuBrettPhos, TrixiePhos, t-BuDavePhos, t-BuMePhos, MePhos, PhDavePhos, VPhos, PhCPhos, XPhos-SO₃Na, water soluble SPhos, CPhos, EtCPhos, RockPhos, AlPhos, and t-Bu PhCPhos.

14. The process of claim 13, wherein the phosphine is selected from tricyclohexylphosphine, XantPhos, DPEPhos, XPhos, SPhos, Cy-JohnPhos, Amphos, and PhDavePhos.

15. The process of claim 12, wherein the phosphite is selected from trimethylphosphite and triphenylphosphite.

16. The process of claim 12, wherein the bis-phosphine is selected from bis(diphenylphosphino)methane (dppm), 1,2′-bis(diphenyl phosphino)ethane (dppe), 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf).

17. The process of claim 16, wherein the bis-phosphine is selected from 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf).

18. The process of claim 12, wherein the metal catalyst is a selected from Pd(dppf)Cl₂, Pd(Amphos)₂Cl₂, Pd(dcypf)Cl₂, Pd(dtbpf)Cl₂, Pd(XantPhos)Cl₂, PdCl₂ (DPEPhos), Pd(PCy₃)Cl₂, XPhos-Pd-G2, and RuPhos-Pd-G2.

19. The process of claim 18, wherein the metal catalyst is Pd(Amphos)₂Cl₂.

20. The process of claim 12, wherein the metal catalyst is a palladacycle.

21. The process of any one of claims 1 to 20, wherein the metal catalyst is formed in solution.

22. The process of any one of claims 1 to 21, wherein the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, Na₂HPO₄, and CsF.

23. The process of claim 22, wherein the suitable base is selected from KOAc, NaHCO₃, and K₂CO₃.

24. The process of any one of claims 1 to 23, wherein the suitable solvent is selected from a polar protic solvent, a polar aprotic solvent, and any combination thereof.

25. The process of any one of claims 1 to 23, wherein the suitable solvent is selected from acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, water, and any combination thereof.

26. The process of claim 25, wherein the suitable solvent is selected from tetrahydrofuran, tetrahydropyran, 1,4-dioxane, 2-methyltetrahydrofuran, cyclopentyl methyl ether, water, and any combination thereof.

27. The process of any one of claims 1 to 26, wherein the coupling conditions include a reaction temperature of about 60° C. to about 100° C.

28. The process of any one of claims 1 to 26, wherein the coupling conditions include a reaction temperature of about 70° C. to about 90° C.

29. The process of claim 28, wherein the coupling conditions include a reaction temperature of about 70° C.

30. The process of any one of claims 1 to 29, wherein the reaction conditions comprise a stir time of about 0.1 h to about 24 h.

31. The process of claim 30, wherein the reaction conditions comprise a stir time of about 0.5 h to about 5 h.

32. The process of any one of claims 1 to 31, wherein the compound of Formula 1 is Compound I, the compound of Formula 2 is Compound II, and the compound of Formula 3 is Compound III, each depicted below:

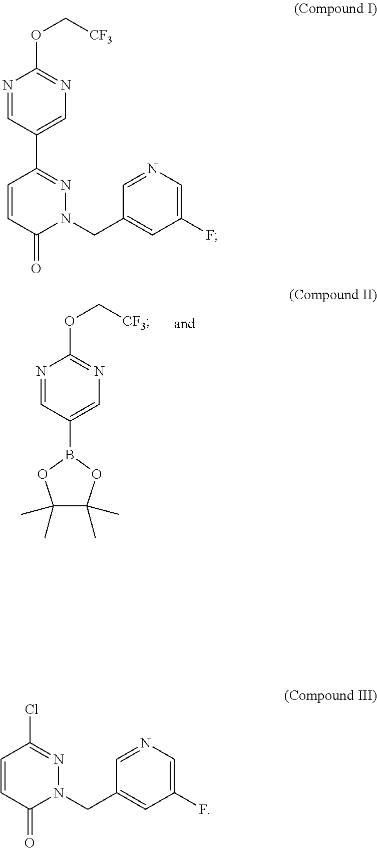 [SMILES: O=c1ccc(-c2cnc(OCC(F)(F)F)nc2)nn1Cc1cncc(F)c1 ; CC1(C)OB(c2cnc(OCC(F)(F)F)nc2)OC1(C)C ; O=c1ccc(Cl)nn1Cc1cncc(F)c1]

33. The process of claim 32, wherein the molar ratio of Compound II to Compound III is from about 1.0:1.0 to about 1.0:1.5.

34. The process of claim 33, wherein the molar ratio of Compound II to Compound III is from about 1.0:1.0 to about 1:1.2.

35. The process of claim 34, wherein the molar ratio of Compound II to Compound III is about 1.0:1.0.

36. The process of any one of claims 32 to 35, wherein the metal catalyst is Pd(Amphos)₂Cl₂.

37. The process of claim 36, wherein the molar ratio of Compound II to the metal catalyst is between about 1.0:0.0001 to about 1.0:0.1.

38. The process of claim 37, wherein the molar ratio of the Compound II to the metal catalyst is between about 1.0:0.001 to about 1.0:0.05.

39. The process of claim 38, wherein the molar ratio of the Compound II to the metal catalyst is between about 1.0:0.05 to about 1.0:0.04.

40. The process of claim 39, wherein the molar ratio of the Compound II to the metal catalyst is between about 1.0:0.01 to about 1.0:0.02.

41. The process of claim 40, wherein the molar ratio of the Compound II to the metal catalyst is about 1.0:0.015.

42. The process of any one of claims 32 to 41, wherein the suitable base is selected from NaHCO₃, K₂CO₃, and Cs₂CO₃.

43. The process of claim 42, wherein the molar ratio of Compound II to the suitable base is from about 1.0:5.0 to about 1:1.0.

44. The process of claim 43, wherein the molar ratio of Compound II to the suitable base is from about 1.0:3.0 to about 1.0:1.0.

45. The process of claim 44, wherein the molar ratio of Compound II to the suitable base is about 1.0:2.0.

46. The process of any one of claims 32 to 45, wherein the solvent is a combination of 2-MeTHF and H₂O.

47. The process of claim 46, wherein the ratio of 2-MeTHF to H₂O is about 2:1.

48. The process of any one of claims 1 to 31, wherein the compound of Formula 3: 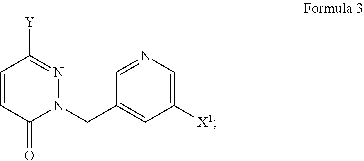
is prepared by contacting a compound of Formula 4: 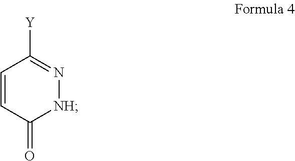
Y is a leaving group;
with a compound of Formula 5: 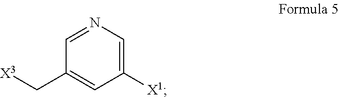
or a salt thereof,
wherein X¹ is a halogen; and
X³ is a halogen;
in the presence of a suitable base, in a suitable solvent, to provide a compound of Formula 3.

49. The process of claim 48, wherein Y is selected from halogen.

50. The process of claim 49, wherein Y is selected from —Cl and —Br.

51. The process of any one of claims 48 to 50, wherein X¹ is selected from —F and —Cl.

52. The process of any one of claims 48 to 51, wherein X³ is selected from —Cl and —Br.

53. The process of any one of claims 48 to 52, wherein the compound of Formula 5 is an acidic salt.

54. The process of claim 53, wherein the compound of Formula 5 is an HCl salt.

55. The process of any one of claims 48 to 54, wherein the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF.

56. The process of claim 55, wherein the suitable base is selected from KOAc, NaHCO₃, and K₂CO₃.

57. The process of any one of claims 48 to 56, wherein the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, methyl-t-butyl ether, water, and any combination thereof.

58. The process of claim 57, wherein the suitable solvent is selected from N-methyl-2-pyrrolidone, tetrahydrofuran, methyl-t-butyl ether, tetrahydropyran, 1,4-dioxane, 2-methyltetrahydrofuran, water, and any combination thereof.

59. The process of any one of claims 48 to 58, wherein the reaction conditions comprise a stir time of about 0.1 h to about 24 h.

60. The process of claim 59, wherein the reaction conditions comprise a stir time of about 0.5 h to about 5 h.

61. The process of any one of claims 48 to 60, wherein the compound of Formula 3 is Compound III, the compound of Formula 4 is Compound IV, and the compound of Formula 5 is Compound V, each depicted below:

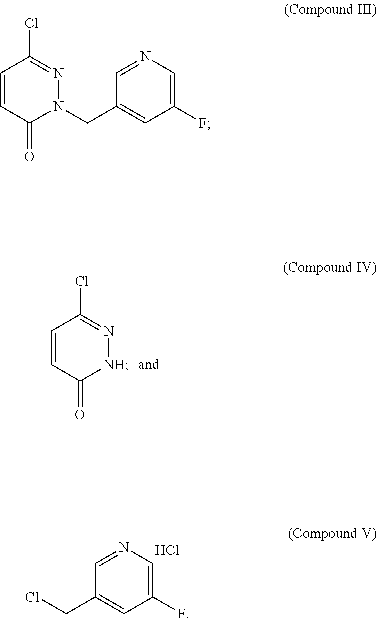 [SMILES: O=c1ccc(Cl)nn1Cc1cncc(F)c1 ; O=c1ccc(Cl)n[nH]1 ; Fc1cncc(CCl)c1 ; Cl]

62. The process of claim 61, wherein the molar ratio of Compound IV to Compound V is from about 1.0:1.0 to about 1.0:1.5.

63. The process of claim 62, wherein the molar ratio of Compound IV to Compound V is from about 1.0:1.0 to about 1.0:1.2.

64. The process of claim 63, wherein the molar ratio of Compound IV to Compound V is about 1.0:1.0.

65. The process of any one of claims 61 to 64, wherein the suitable base is K₂CO₃.

66. The process of claim 65, wherein the molar ratio of Compound IV to the suitable base is from about 1.0:5.0 to about 1.0:1.0.

67. The process of claim 65, wherein the molar ratio of Compound IV to the suitable base is from about 1.0:4.0 to about 1.0:2.0.

68. The process of claim 65, wherein the molar ratio of Compound IV to the suitable base is about 1.0:3.0.

69. The process of any one of claims 61 to 68, wherein the solvent is a combination of NMP and H₂O.

70. The process of claim 69, wherein the solvent combination is 20% H₂O by volume.

71. The process of any one of claims 61 to 68, wherein the solvent is a combination of NMP 2-methyl THF and H₂O.

72. The process of claim 71, wherein the solvent combination is 50% H₂O by volume.

73. The process of any one of claims 1 to 72, the process further comprising the preparation of a compound of Formula 2: 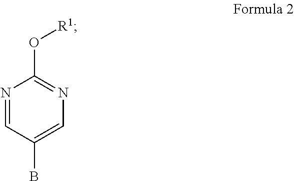
wherein R¹ is C₁-C₆ haloalkyl; and
B is selected from a boronic acid and boronic ester;
comprising:
contacting a compound of Formula 6: 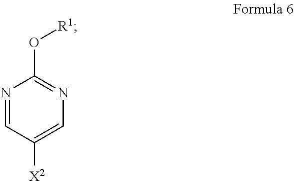
wherein R¹ is C₁-C₆ haloalkyl; and
X² is halogen;
with a boron compound, wherein the boron compound comprises a boron-boron bond or a boron-hydrogen bond;
in the presence of a metal catalyst, a suitable base, in a suitable solvent, to provide a compound of Formula 2.

74. A process for the preparation of a compound of Formula 2: 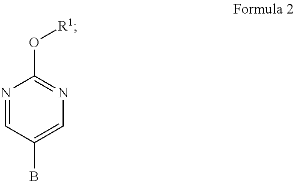
wherein R¹ is C₁-C₆ haloalkyl; and
B is selected from a boronic acid and boronic ester;
comprising:
contacting a compound of Formula 6: 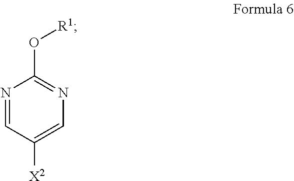
wherein R¹ is C₁-C₆ haloalkyl; and
X² is halogen;
with a boron compound, wherein the boron compound comprises a boron-boron bond or a boron-hydrogen bond;
in the presence of a metal catalyst, a suitable base, in a suitable solvent, to provide a compound of Formula 2.

75. The process of claim 73 or 74, wherein R¹ is C₁-C₃ haloalkyl.

76. The process of claim 75, wherein R¹ is selected from —CF₃, —CHF₂, —CH₂CF₃, and —CH₂CHF₂.

77. The process of any one of claims 71 to 74, wherein B is selected from

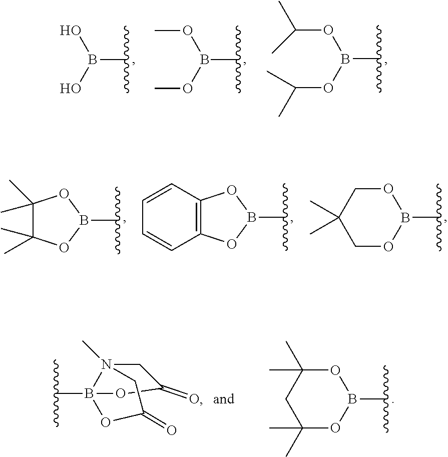

78. The process of claim 77, wherein B is selected from

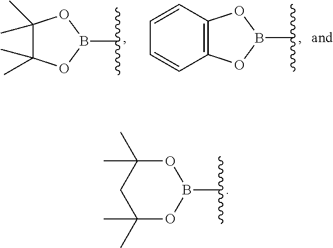

79. The process of any one of claims 73 to 78, wherein X² is selected from —Cl, —Br, and —I.

80. The process of any one of claims 73 to 79, wherein the boron compound is selected from

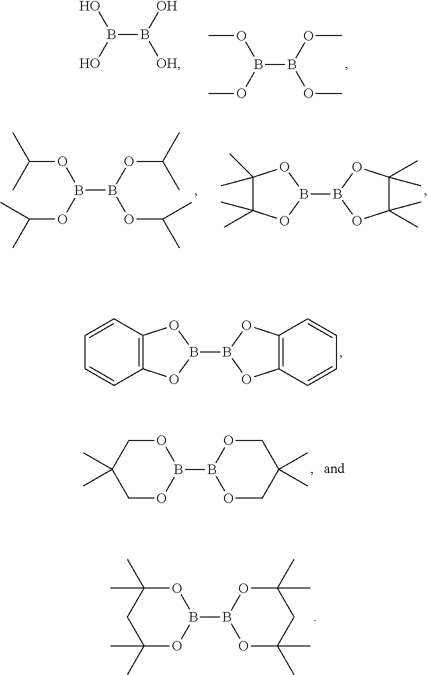

81. The process of claim 80, wherein the boron compound is selected from,

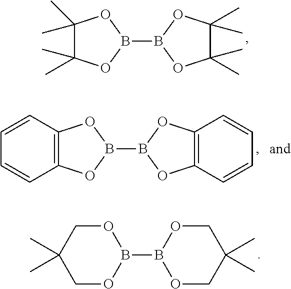

82. The process of any one of claims 73 to 81, wherein the metal catalyst is a palladium catalyst.

83. The process of any one of claims 73 to 82, wherein the metal catalyst is selected from a palladium (0) or palladium (II) catalyst.

84. The process of any one of claims 73 to 83, wherein the metal catalyst comprises palladium and one or more ligand, wherein the one or more ligand is selected from a phosphine, phosphite, and a bis-phosphine.

85. The process of claim 84, wherein the phosphine is selected from trimethyl phosphine, tricyclohexylphosphine, tri-(tert-butyl)-phosphine, XantPhos, DPEPhos, XPhos, SPhos, JohnPhos, Cy-JohnPhos, Amphos, triphenylphosphine, methyldiphenylphosphine, Me4 t-BuXphos, t-BuXPhos, t-BuXantPhos, RuPhos, DavePhos, sSPhos, AdBrettPhos, BrettPhos, JackiePhos, t-BuBrettPhos, TrixiePos, t-BuDavePhos, t-BuMePhos, MePhos, PhDavePhos, VPhos, PhCPhos, XPhos-SO₃Na, water soluble SPhos, CPhos, EtCPhos, RockPhos, AlPhos, t-Bu PhCPhos, AlPhos.

86. The process of claim 85, wherein the phosphine is selected from tricyclohexylphosphine, XantPhos, DPEPhos, XPhos, SPhos, Cy-JohnPhos, Amphos, and PhDavePhos.

87. The process of claim 84, wherein the phosphite is selected from trimethylphosphite and triphenylphosphite.

88. The process of claim 84, wherein the bis-phosphine is selected from bis(diphenylphosphino)methane (dppm), 1,2′-bis(diphenyl phosphino)ethane (dppe), 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf).

89. The process of claim 88, wherein the bis-phosphine is selected from 1,1′-bis(diphenylphosphino)ferrocene (dppf), 1,1′-bis(di-cyclohexylphosphino)ferrocene (dcypf), 1,1′-bis(di-tert-butylphosphino)ferrocene (dtbpf), and 1,1′-bis(di-isopropylphosphino)ferrocene (dippf).

90. The process of claim 84, wherein the metal catalyst is a selected from Pd(dppf)Cl₂, Pd(Amphos)₂Cl₂, Pd(dcypf)Cl₂, Pd(dtbpf)Cl₂, Pd(XantPhos)Cl₂, PdCl₂ (DPEPhos), Pd(PCy₃)Cl₂, XPhosPd G2, and RuPhos-Pd-G2.

91. The process of claim 84, wherein the metal catalyst is Pd(dppf)Cl₂.

92. The process of claim 84, wherein the metal catalyst is Pd(Amphos)₂Cl₂.

93. The process of any one of claims 73 to 92, wherein the metal catalyst is formed in solution.

94. The process of any one of claims 73 to 90, wherein the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF.

95. The process of claim 91, wherein the suitable base is selected from KOAc, NaHCO₃, and K₂CO₃.

96. The process of any one of claims 73 to 95, wherein the suitable solvent is selected from a polar protic solvent, a polar aprotic solvent, and any combination thereof.

97. The process of any one of claims 73 to 96, wherein the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, water, and any combination thereof.

98. The process of any one of claims 73 to 96, wherein the suitable solvent is selected from N-methyl-2-pyrrolidone, acetonitrile, dimethylformamide, diethyl ether, ethanol, tetrahydrofuran, 2-methyltetrahydrofuran, tetrahydropyran, isopropyl alcohol, 1,4-dioxane, toluene, cyclopentyl methyl ether, and any combination thereof.

99. The process of claim 97 or 98, wherein the suitable solvent is selected from N-methyl-2-pyrrolidone, tetrahydrofuran, tetrahydropyran, 1,4-dioxane, 2-methyltetrahydrofuran, water, and any combination thereof.

100. The process of claim 99, wherein the suitable solvent is 2-methyltetrahydrofuran.

101. The process of any one of claims 73 to 100, wherein the borylation reaction conditions comprise a reaction temperature of about 60° C. to about 100° C.

102. The process of claim 101, wherein the borylation reaction conditions comprise a reaction temperature of about 70° C. to about 90° C.

103. The process of claim 102, wherein the borylation reaction conditions comprise a reaction temperature of about 85° C.

104. The process of claim of claim 98, wherein any water present in the suitable solvent is reduced.

105. The process of claim 104, wherein the suitable solvent is 2-methyltetrahydrofuran and any water in the suitable solvent is reduced by azeotropic distillation.

106. The process of claim 104, wherein the boronic ester or boronic acid, the suitable base, and the suitable solvent are heated to reduce the water from the solvent prior to the addition of the metal catalyst and a compound of Formula 2.

107. The process of claims 104 to 106, wherein the removal of water decreases the formation of a homocoupling impurity of Formula 6 by between 1 to 10%.

108. The process of claim 107, wherein the removal of water decreases the formation of a homocoupling impurity of Formula 6 by between 1 to 5%.

109. The process of claims 104 to 108, wherein the removal of water increases the yield of the compound of Formula 2 by 1 to 10%.

110. The process of claim 109, wherein the removal of water increases the yield of the compound of Formula 2 by 1 to 5%.

111. The process of any one of claims 73 to 110, wherein the reaction conditions comprise a stir time of about 0.1 h to about 48 h.

112. The process of claim 111, wherein the reaction conditions comprise a stir time of about 0.5 h to about 24 h.

113. The process of any one of claims 73 to 112, wherein the compound of Formula 2 is Compound II, the compound of Formula 6 is Compound VI, and each depicted below: 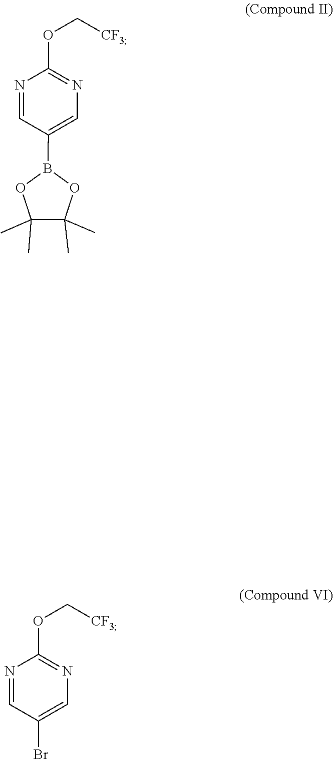 [SMILES: CC1(C)OB(c2cnc(OCC(F)(F)F)nc2)OC1(C)C ; FC(F)(F)COc1ncc(Br)cn1]
and the boron compound is

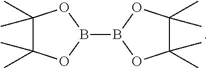

114. The process of claim 113, wherein the molar ratio of Compound VI to the boron compound is from about 1.0:1.0 to about 1.0:1.5.

115. The process of claim 114, wherein the molar ratio of Compound VI to the boron compound is from about 1.0:1.0 to about 1:1.2.

116. The process of claim 115, wherein the molar ratio of Compound VI to the boron compound is about 1.0:1.0.

117. The process of any one of claims 113 to 116, wherein the suitable base is KOAc.

118. The process of claim 113, wherein the molar ratio of Compound VI to the suitable base is from about 1.0:5.0 to about 1.0:1.0.

119. The process of claim 118, wherein the molar ratio of Compound VI to the suitable base is from about 1.0:4.0 to about 1.0:2.0.

120. The process of claim 119, wherein the molar ratio of Compound VI to the suitable base is about 1.0:3.0.

121. The process of any one of claims 113 to 120, wherein the metal catalyst is Pd(dppf) 2Cl₂.

122. The process of claim 121, wherein the molar ratio of Compound VI to the metal catalyst is between about 1.0:0.0001 to about 1.0:0.1.

123. The process of claim 122, wherein the molar ratio of the Compound VI to the metal catalyst is between about 1.0:0.001 to about 1.0:0.05.

124. The process of claim 122, wherein the molar ratio of the Compound VI to the metal catalyst is between about 1.0:0.05 to about 1.0:0.04.

125. The process of claim 122, wherein the molar ratio of the Compound VI to the metal catalyst is between about 1.0:0.01 to about 1.0:0.02.

126. The process of claim 122, wherein the molar ratio of the Compound VI to the metal catalyst is about 1.0:0.015.

127. The process of any one of claims 113 to 126, wherein the solvent is 2-MeTHF.

128. The process of any one of claims 73 to 127, wherein the compound of Formula 6: 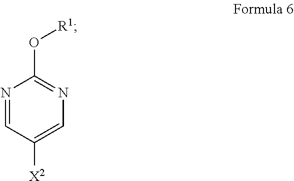
is prepared by contacting a compound of Formula 7: 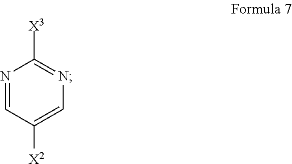
wherein X² is a halogen; and
X³ is halogen;
with a compound of Formula 8: 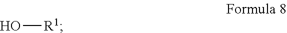
wherein R¹ is C₁-C₆ haloalkyl;
in the presence of a suitable base, in a suitable solvent, to provide a compound of Formula 6.

129. The process of claim 128, wherein R¹ is C₁-C₃ haloalkyl.

130. The process of claim 129, wherein R¹ is selected from —CF₃, —CHF₂, —CH₂CF₃, and —CH₂CHF₂.

131. The process of any one of claims 128 to 130, wherein X² is selected from —Cl, —Br, and —I.

132. The process of any one of claims 128 to 131, wherein X³ is selected from —Cl, and —Br.

133. The process of any one of claims 128 to 132, wherein the suitable base is selected from triethylamine, diisopropylethylamine, 1,2,2,6,6-pentamethylpiperidine, tributylamine, 1,8-diazabicycloundec-7-ene (DBU), NaHCO₃, NaOAc, KOAc, KOMe, KOtBu Ba(OH)₂, Li₂CO₃, Na₂CO₃, K₂CO₃, KHCO₃, Cs₂CO₃, Na₃PO₄, K₃PO₄, KF, and CsF.

134. The process of claim 133, wherein the suitable base is selected from KOAc, NaHCO₃, and K₂CO₃.

135. The process of any one of claims 128 to 134, wherein the suitable solvent is selected from a polar protic solvent, a polar aprotic solvent, and any combination thereof.

136. The process of any one of claims 128 to 135, wherein the suitable solvent is selected from acetonitrile, dimethyl sulfoxide, dimethylformamide, diethyl ether, ethanol, tetrahydrofuran, 2-methyltetrahydrofuran, tetrahydropyran, N-methyl-2-pyrrolidone, methyl-t-butyl ether, isopropyl alcohol, 1,4-dioxane, toluene, cyclopentyl methyl ether, water, and any combination thereof.

137. The process of claim 136, wherein the suitable solvent is selected from acetonitrile, dimethyl sulfoxide, tetrahydrofuran, N-methyl-2-pyrrolidone, dimethylformamide, methyl-t-butyl ether, 1,4-dioxane, water, and any combination thereof.

138. The process of any one of claims 128 to 137, wherein the compound of Formula 6 is Compound VI, the compound of Formula 7 is Compound VII, each depicted below: 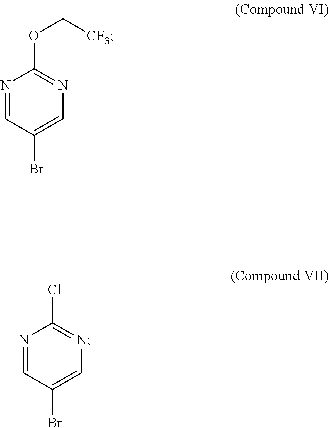 [SMILES: FC(F)(F)COc1ncc(Br)cn1 ; Clc1ncc(Br)cn1]
and the compound of Formula 8 is CF₃CH₂OH.

139. The process of claim 138, wherein the molar ratio of Compound VII to CF₃CH₂OH is from about 1.0:1.0 to about 1.0:1.5.

140. The process of claim 139, wherein the molar ratio of Compound VII to CF 3CH₂OH is from about 1.0:1.0 to about 1.0:1.2.

141. The process of claim 140, wherein the molar ratio of Compound VII to CF 3CH₂OH is about 1.0:1.1.

142. The process of any one of claims 138 to 141, wherein the suitable base is K₂CO₃.

143. The process of claim 142, wherein the molar ratio of Compound VII to the suitable base is from about 1.0:5.0 to about 1.0:1.0.

144. The process of claim 143, wherein the molar ratio of Compound VII to the suitable base is from about 1.0:4.0 to about 1.0:1.0.

145. The process of claim 144, wherein the molar ratio of Compound VII to the suitable base is about 1.0:1.6.

146. The process of any one of claims 138 to 145, wherein the solvent is dimethylformamide.

147. The process of any one of claims 138 to 145, wherein the solvent is N-methyl-2-pyrrolidone.
